(19) United States
(12) Patent Application Publication
(10) Pub. No.: US 2023/0218900 A1
(43) Pub. Date: Jul. 13, 2023

(54) CLOSED LOOP SYSTEMS AND METHODS FOR MANAGING PAIN OF A PATIENT

(71) Applicant: Advanced Neuromodulation Systems, Inc., Plano, TX (US)

(72) Inventors: Hyun-Joo Park, Frisco, TX (US); Yagna Pathak, Skokie, IL (US); Hongxuan Zhang, Austin, TX (US); Anahita Kyani, Plano, TX (US); Erika Ross, Dallas, TX (US); Jeffrey Urbanski, Frisco, TX (US)

(21) Appl. No.: 17/574,542

(22) Filed: Jan. 12, 2022

(51) Int. Cl. A61N 1/36; A61N 1/375

(52) U.S. Cl. CPC A61N 1/36071; A61N 1/36062; A61N 1/36139; A61N 1/375

(57) ABSTRACT

Devices and methods to effectuate closed loop electrical stimulation of nerve tissue, based on feedback data, to mitigate pain of a patient are disclosed. Feedback data corresponding to bioelectric signals of neurons stimulated by stimulation pulses may be received and analyzed. Based on receipt of the feedback data, it may be determined to modify one or more stimulation parameters, corresponding to the stimulation pulses, to enhance an efficacy of the stimulation pulses at blocking generation and/or propagation of one or more pain signals through a neuroanatomy of the patient. Subsequent and additional stimulation pulses may be provided based on a modified set of stimulation parameters and configured to enhance attenuation of generation and/or transmission of pain signals through the neuroanatomy of the patient to ultimately reduce a level of pain experienced by the patient.

### TECHNICAL FIELD

The present invention relates generally to neurostimulation systems and more specifically, to closed loop neurostimulation systems leveraging high electrode density leads to sense neuronal activity and to deliver electrical stimulation in connection with managing patient pain or other conditions of a patient.

### BACKGROUND OF THE INVENTION

Implantable medical devices (IMDs) may be implanted within a patient's body and provide functionality to treat a wide variety of medical conditions. For example, IMDs may be used to control delivery of electrical stimulation pulses or signals to a targeted tissue (e.g., brain tissue, muscle tissue, nerves, etc.) of a patient to treat pain, movement disorders (e.g., Parkinson's disease), epilepsy and seizures, or other conditions of the patient (e.g., cardiac pace making, cardiac rhythm management, treatments for congestive heart failure, implanted defibrillators, incontinence, depression, and the like). The IMDs generally include an implantable pulse generator (IPG) that generates electrical pulses or signals that are transmitted to a targeted tissue or nerves through a therapy delivery element, such as a lead having one or more electrodes. The therapy delivery element is generally placed within the patient's body to achieve therapeutic efficacy or reduced side effects. For example, therapy delivery elements in the form of leads are commonly implanted along peripheral nerves, within the epidural or intrathecal space of the spinal column, and around the heart, brain, or other organs or tissue of a patient. Once implanted, the lead extends from the stimulation site to the location of the implantable electrical stimulation device. The distance from the stimulation site to the IMD may, for example, be on the order of 20-100 cm. In some situations, a lead extension may be utilized between a lead and IMD to span relatively long distances.

Leads (e.g., stimulation leads) configured for use with IMDs typically include a connector apparatus (e.g., one or more electrical contacts configured to connect electrically couple the lead to the IMD disposed on a proximal end and the aforementioned electrodes (e.g., one or more electrically conductive rings, split or non-continuous rings, etc.) disposed on a distal end. Conductive wires interconnect the electrodes at the distal end to corresponding contacts of the connector apparatus at a proximal end. The conductive wires are usually surrounded by an insulating material to electrically isolate the conductive wires from each other. An insulating or protective jacket (e.g., a flexible, resilient member formed biocompatible polymer) may surround the body of leads such that the conductive wires are disposed within the jacket and protected from body tissue, fluids, and the like.

Traditionally, the aforementioned electrodes are principally deployed for providing electrical stimulation to one or more parts of an anatomy of a patient. For example, in pain management applications electrical stimulation may be provided via electrodes disposed proximate to tissue of the patient's spinal cord (e.g., spinal cord stimulation (SCS)), such as a dorsal root ganglion (DRG). A DRG is a cluster of neurons in a dorsal root of a spinal nerve. While clinical data suggests that providing electrical stimulation to the DRG might mitigate pain, adjusting one or more stimulation parameters of the electrical stimulation pulses associated with the electrical stimulation has proved challenging due, in part, to an inability to measure the stimulation effect by collecting adequate sensory data after delivery of the electrical stimulation pulses. For example, existing technologies for detecting sensory data in connection with SCS suffer from degraded signal quality attributable to factors such as stimulation artifacts. Lack of reliable sensory data has impeded use of closed loop control of neurostimulation devices for certain types of therapies, such as pain management via SCS. An additional challenge is that electrodes are usually positioned to optimize lead manufacturability and not necessarily to optimize delivery of electrical stimulation pulses to particular anatomical regions of a patient or to provide stimulation pulse capabilities specific to a target region of the patient's anatomy. As such, existing technologies for recording sensory data may be insufficient to record sensory data suitable for use in closed loop systems (e.g., due to noise, artifacts, etc.).

### BRIEF SUMMARY OF THE INVENTION

In certain embodiments, a neurostimulation system for managing pain of a patient in a closed loop manner is provided. The neurostimulation system includes an IMD electrically coupled to a lead (e.g., a stimulation lead) that includes a lead body having a plurality of electrodes disposed at a distal end. The electrodes may be distributed along a length of the lead body and configured to deliver electrical stimulation pulses (hereinafter “stimulation pulses”) to target tissue of the patient, such as spinal tissue or epineural tissue of the patient. The plurality of electrodes may include sensing electrodes (e.g., electrodes configured to record or sense signals generated by tissue of the patient) stimulation electrodes (e.g., electrodes configured to deliver stimulation pulses to target tissue of the patient), and/or electrodes configured to both sense signals generated by tissue of the patient and deliver stimulation pulses to target tissue of the patient. The electrodes may be positioned and arranged along the length the lead body configured to enhance the electrode sensing (e.g., to increase a signal to noise ratio (SNR), increase the sensitivity at which signals may be sensed, etc.) and to provide increased control over the delivery of stimulation pulses to the target tissue.

In particular, the neurostimulation system is implanted in the patient and includes the stimulation lead with a first plurality of electrodes, a second plurality of electrodes, and a third plurality of electrodes. The second plurality of electrodes is disposed adjacent to a DRG of the patient, and the first plurality of electrodes is disposed adjacent to the dorsal root or rootlet between the DRG and a spinal cord of the patient (also referred to as a central process of the pseudo-unipolar sensory neuron). The third plurality of electrodes is disposed away from the DRG and the spinal cord and adjacent to a spinal nerve (also referred to as a peripheral process of the pseudo-unipolar sensory neuron). Additionally, the neurostimulation system includes an IPG.

In an aspect of the disclosure, a method of providing a neurostimulation therapy to a patient using the neurostimulation system is presented. The method may include sensing, via the second plurality of electrodes, activity of nociceptive neurons of the DRG. The activity of the nociceptive neurons may be indicative of neuropathic pain of the patient. Additionally, the method may include switching, by a controller (e.g., of the neurostimulation system), an operating mode of the neurostimulation system between a first operating mode and a second operating mode based on the sensing the activity of the nociceptive neurons of the DRG. The first operating mode may include sensing the activity of the nociceptive neurons at least partially simultaneously with delivery of one or more stimulation pulses to the patient and the second operating mode may include performing the sensing in between delivery of the one or more stimulation pulses to the patient. Moreover, the method may include generating the one or more stimulation pulses using the IPG, and applying the one or more stimulation pulses in accordance with the first operating mode or the second operating mode.

In another aspect of the disclosure, a further method of providing a neurostimulation therapy to a patient using the neurostimulation system is disclosed. The method includes delivering, via the second plurality of electrodes, one or more stimulation pulses to the DRG of the patient. The first one or more stimulation pulses are generated by the IPG based on stimulation parameters configured to mitigate pain of the patient. Additionally, the method includes sensing, via the first plurality of electrodes, first electroneurogram (ENG) data corresponding to the neuronal activity of the neural tissue disposed between the DRG and the spinal cord of the patient and adjacent to the dorsal root and rootlets. Moreover, the method includes sensing, via the third plurality of electrodes, second ENG data corresponding to the neuronal activity of the neural tissue disposed away from the DRG and adjacent to the spinal nerve from which the DRG emerges. Further, the method includes estimating, by a controller (e.g., of the neurostimulation system), a blocking effect of the first one or more stimulation pulses delivered to the DRG of the patient based on the first ENG data and the second ENG data. Moreover, the method includes generating second one or more stimulation pulses using the IPG and based on the blocking effect. Additionally, the method includes applying the second one or more stimulation pulses to the DRG using one or more electrodes of the second plurality of electrodes.

In yet another aspect of the disclosure, a further method of providing a neurostimulation therapy to the patient using the neurostimulation system is disclosed. The method includes sensing, by first one or more electrodes of the first plurality of electrodes, evoked compound action potential (ECAP) signals induced by one or more stimulation pulses delivered by second one or more electrodes of the third plurality of electrodes adjacent to the spinal nerve of the patient. Further, the method includes estimating, based on ECAP data corresponding to the ECAP signals, a blocking effect of second one or more stimulation pulses delivered to the DRG by one or more electrodes of the second plurality of electrodes. Moreover, the method includes generating third one or more stimulation pulses using the IPG based on sensing the ECAP signals and applying the third one or more stimulation pulses to the DRG via one or more of the second plurality of electrodes.

In a further aspect of the disclosure, an additional method is disclosed for providing a neurostimulation therapy to a patient using a neurostimulation system. The method includes selecting, by a controller of the IMD, a therapy modality for conducting a closed-loop neurostimulation therapy. The IMD is programmed to perform a plurality of closed-loop neurostimulation therapies and the therapy modality is selected from among the plurality of closed-loop neurostimulation therapies programmed for the IMD. The plurality of closed-loop neurostimulation therapies include at least a first therapy modality in which first one or more stimulation pulses are delivered to the DRG, via first one or more electrodes of the second plurality of electrodes, based on sensed activity of nociceptive neurons. Moreover, the closed-loop neurostimulation therapies include a second therapy modality in which second one or more stimulation pulses are delivered to the DRG, via the one or more electrodes of the second plurality of electrodes, based on first electroneurogram data associated with first neural tissue disposed between the DRG and the spinal cord of the patient and adjacent to the dorsal root or rootlets of the patient and second electroneurogram data associated with second neural tissue disposed away from the DRG and adjacent to a spinal nerve from which the DRG emerges. Further, the closed-loop neurostimulation therapies include a third therapy modality in which third one or more stimulation pulses are delivered to the DRG, via the one or more electrodes of the second plurality of electrodes, based on ECAP data corresponding to ECAP signals sensed at first one or more electrodes of the first plurality of electrodes. Additionally, the method includes generating the first one or more stimulation pulses, the second one or more stimulation pulses, the third one or more stimulation pulses, or any combination thereof using the IPG and based on the therapy modality. Moreover, the method includes applying, via one or more electrodes, the first one or more stimulation pulses, the second one or more stimulation pulses, the third one or more stimulation pulses, or any combination thereof to neural tissue of the patient.

The neurostimulation system leverages closed loop techniques to dynamically monitor and adjust the electrical stimulation delivered to the patient. To illustrate, the IMD may include an IPG configured to deliver stimulation pulses to spinal tissue of a patient via one or more electrodes of the plurality of electrodes. The stimulation pulses may be generated based on stimulation parameters configured to mitigate pain of a patient. The IMD may also include a controller configured to receive feedback data from particular electrodes of the plurality of electrodes, such as electrodes disposed proximate particular spinal and/or epineural tissue (e.g., a central process, a DRG, a peripheral process, and the like), and to determine whether to modify the stimulation parameters based, at least in part, on the feedback data. In some aspects, the controller may determine whether to modify the stimulation parameters based on analysis of the feedback data. For example, the controller may analyze the feedback data to determine a state (e.g., a mobility state, a level of pain or a pain state, etc.) of the patient, and the determination to modify (or not modify) the stimulation parameters may be based on the state. Upon determining to modify the stimulation parameters, the controller may modify one or more of the stimulation parameters to produce a modified set of stimulation parameters configured to improve or enhance a state of the patient, such as to decrease a pain state of the patient. The IPG utilizes the modified set of stimulation parameters to generate additional stimulation pulses for subsequent delivery to the spinal tissue and/or epineural tissue of the patient.

It is noted that utilizing closed loop techniques may be particularly beneficial to patients experiencing pain. For example, the amount of pain the patient experiences may be dependent on a mobility state of the patient (e.g., is the patient sitting, standing, lying down, running, walking, etc.) or other factors, which may change over time. Implementing closed loop techniques in accordance with aspects of the present disclosure enables the parameters used to control the stimulation therapy to be automatically adjusted according to a current state of the patient (e.g., the patient's pain state, mobility state, etc.), which may improve the overall therapeutic effect of the therapy and more effectively mitigate the pain of the patient as compared to previous systems that utilized a static set of stimulation parameters that may be effective at mitigating pain in some but not all circumstances.

In an aspect, the disclosed closed loop systems for treating pain of a patient may be configured to detect biomarkers indicative of a pain level of the patient based on the feedback data. For example, sensing electrodes of an implanted lead may collect or record feedback data (e.g., neuronal data corresponding to a peripheral process and a central process of the sensory neurons of the patient, activity of nociceptive neurons of the DRG, evoked compound action potentials (ECAPs) of the central process and/or DRG). The controller may analyze the feedback data to determine whether a biomarker indicative of the patient's pain state is present, and may determine whether to adjust one or more stimulation parameters used to treat the patient's pain state based on the presence (or absence) of the biomarker(s). To illustrate, the feedback data may include neuronal data corresponding to pain signals originating from peripheral nerves or other tissue of the patient.

In an aspect, the controller may detect the presence of a biomarker indicative of the patient's pain state based on analysis of the neuronal data, such as a biomarker indicative of a blocking effect achieved via stimulation of the DRG. When present, the biomarker may indicate a level of pain experienced by the patient (e.g., a higher blocking effect may indicate a lower patient pain level and a lower blocking effect may indicate a higher patient pain level). In an additional or alternative aspect, a biomarker indicative of a level of patient pain may be detected based on the feedback data that includes activity of nociceptive neurons in the DRG. The controller may analyze the feedback data to detect changes in the nociceptive neuron activity (e.g., changes indicative of hyperactivity or abnormally elevated activity), where the changes in nociceptive neuron activity may provide a biomarker indicative of the patient's pain state or level. The nociceptive neuron activity may be collected or recorded simultaneously with stimulation of patient tissue. However, stimulation artifacts caused by the stimulation of the patient tissue may degrade the ability of the controller to detect changes in the nociceptive neuron activity with sufficient accuracy to identify biomarkers indicative of pain. In such instances, the controller may modify the stimulation parameters to control stimulation of patient's tissue and sensing or recording of the feedback data sequentially, rather than simultaneously. In yet another additional or alternative aspect, biomarkers indicative of patient pain levels may also be detected based on feedback data that include recorded or sensed ECAPs. In particular, the controller may analyze the ECAPs to estimate the blocking effect provided by stimulation of the DRG.

As described above, the blocking effect provided by stimulation of the DRG may serve as a biomarker indicative of a patient pain level. The controller may use the above-described biomarkers to manage a stimulation therapy for treating patient pain in a closed loop manner. For example, the controller may utilize the detected biomarkers and/or characteristics of the biomarkers (e.g., changes to the biomarker over time or information derived from the biomarkers, such as an estimated pain level) to adjust or modify one or more stimulation parameters, where the adjustments or modifications are configured to enhance the therapeutic effect of the stimulation therapy (i.e., reduce the amount of pain perceived by the patient).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1 shows a block diagram illustrating aspects of a neurostimulation system according to embodiments of the present disclosure;

FIG. 2 illustrates a stimulation and sensing lead placed transforaminally according to embodiments of the present disclosure;

FIG. 3 shows a block diagram of a stimulation and sensing lead having a plurality of electrodes for sensing and/or stimulation according to embodiments of the present disclosure;

FIG. 4 is a flowchart depicting an exemplary process for providing closed loop electrical stimulation to mitigate pain of a patient according to embodiments of the present disclosure;

FIG. 5 is a flowchart depicting an exemplary process for determining whether to modify one or more stimulation parameters based, at least in part, on feedback data according to embodiments of the present disclosure;

FIG. 6 is a flowchart depicting another exemplary process for providing closed loop electrical stimulation to mitigate pain of a patient according to embodiments of the present disclosure;

FIG. 7 is a flowchart depicting yet another exemplary process for providing closed loop electrical stimulation therapy to mitigate pain of a patient according to embodiments of the present disclosure;

FIG. 8 is a flowchart depicting a further exemplary process for providing closed loop electrical stimulation therapy to mitigate pain of a patient according to embodiments of the present disclosure;

FIGS. 9A and 9B illustrate a process for manufacturing a lead having high density electrodes for sensing and/or stimulation according to embodiments of the present disclosure; and

FIGS. 10A and 10B illustrate an additional process for manufacturing a lead having high density electrodes for sensing and/or stimulation according to embodiments of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION OF THE INVENTION

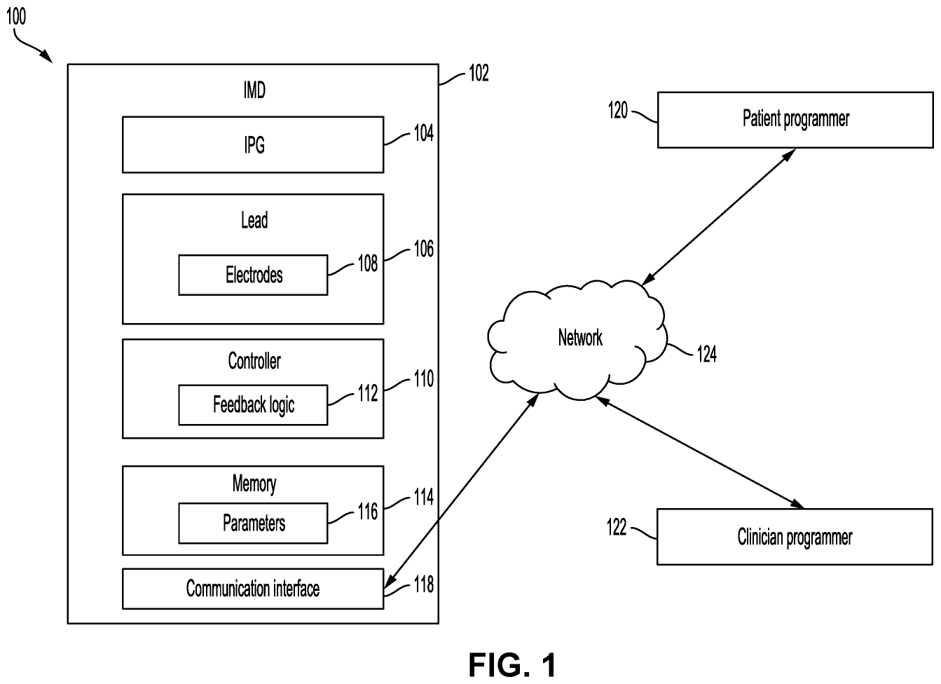

Referring to FIG. 1, a block diagram illustrating aspects of a neurostimulation system according to embodiments of the present disclosure is shown as system 100. System 100 may be deployed to provide closed loop electrical stimulation, such as may be delivered to spinal tissue of a patient to mitigate pain. As shown in FIG. 1, system 100 includes IMD 102, patient programmer 120, and clinician programmer device 122. IMD 102 may be configured to communicate with patient programmer 120 and/or clinician programmer device 122 via network 124.

IMD 102 includes IPG 104, lead 106 (e.g., a stimulation and sensing lead), controller 110, memory 114, and communication interface 118. IPG 104 may include electronics, such as analog to digital converters (ADCs), digital to analog converters (DACs), filters, etc., configured to generate one or more electrical pulses in accordance with a set of stimulation parameters. The stimulation parameters may be configured by inputs or information, such as provided to IMD 102 by patient programmer device 120, clinician programmer device 122, or both via network 124, to achieve a particular therapeutic effect when the one or more stimulation pulses are delivered to tissue of a patient. IPG 104 may be coupled to lead 106, controller 110, or both.

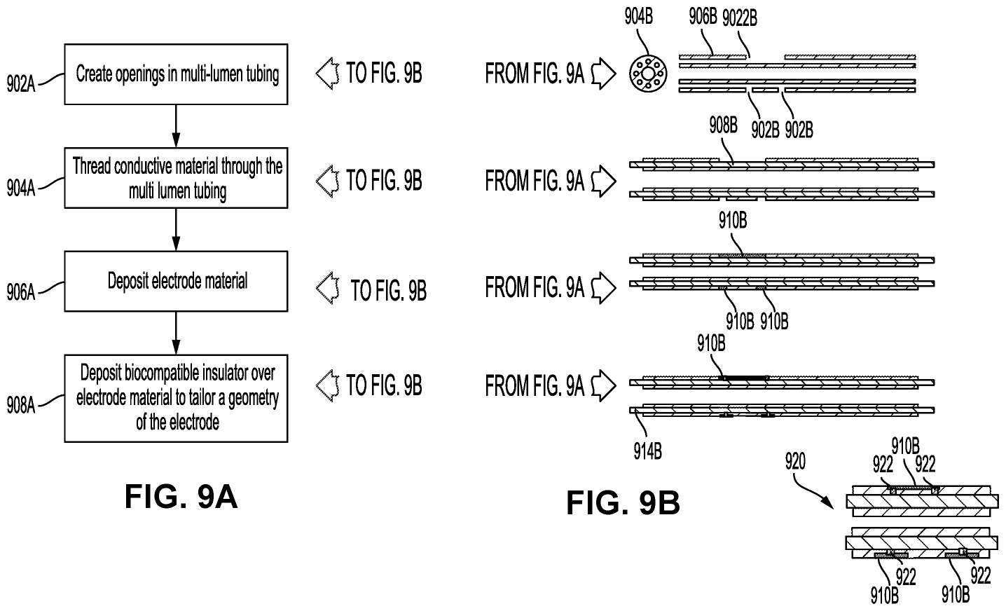

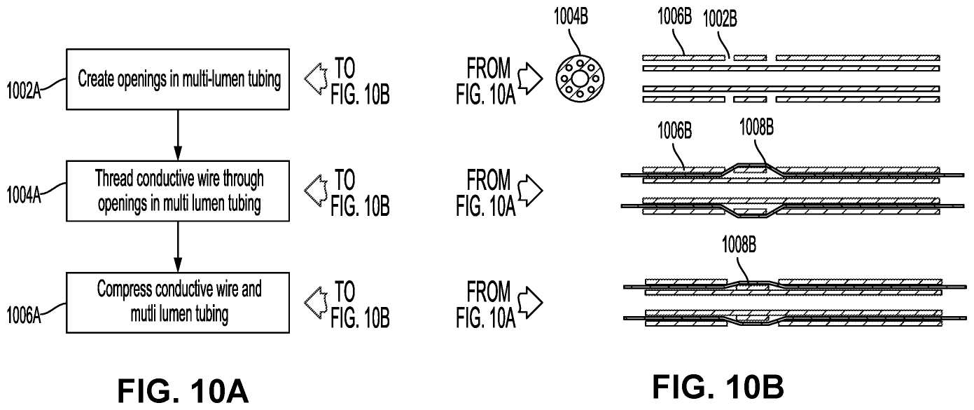

Lead 106 may be coupled to IMD 102 to enable stimulation pulses (e.g., the electrical pulses generated by IPG 104) to be delivered to tissue of the patient via electrodes 108. Lead 106 includes a lead body having electrically conductive wires disposed therein. In some aspects, insulative material may surround the electrically conductive wires within the lead body of lead 106. Additionally, lead 106 may include an insulative or protective jacket surrounding the electrically conductive wires and the insulative material (if provided). The jacket may be formed from a biocompatible polymeric material, such as polyethylene, polypropylene, etc. to protect the lead wires and other components from fluids or other agents when lead 106 is implanted within the patient's body. In some aspects, lead jacket of lead 106 may include a plurality of openings through which one or more electrodes 108 may be exposed, as explained more fully below with reference to FIGS. 9A-10B. In embodiments, lead 106 may be configured to be positioned within target anatomy of a patient, such as a region of the spine or another location.

Lead 106 may include a plurality of electrodes, such as electrodes 108. Electrodes 108 may include sensing electrodes, stimulation electrodes, or sensing and stimulation electrodes. Generally, sensing electrodes may be configured to perform sensing operations, such as receiving or sensing signals (e.g., bioelectrical signals) generated by neural tissue of a patient, such as neuronal activity generated at the dorsal root ganglion of the patient. Stimulation electrodes may be configured to provide stimulation pulses to the neural tissue but may not be configured to perform sensing. Sensing and stimulation electrodes may be configured to both stimulate neural tissue and to sense neuronal activity associated with the stimulated neural tissue. In some aspects, electrodes 108 include sensing electrodes and stimulation electrodes, but not sensing and stimulation electrodes. In additional or alternative aspects, electrodes 108 may all be sensing and stimulation electrodes. In another additional or alternative aspect, electrodes 108 may include sensing and stimulation electrodes and stimulation electrodes. It is noted that while electrodes 108 have been described above as including specific arrangements or combinations of sensing, stimulation, and sensing and stimulation electrodes, such description has been provided for purposes of illustration, rather than by way of limitation and that other combinations and arrangements of electrodes and electrode types may be utilized by embodiments of the present disclosure.

Additionally, one or more of electrodes 108 may be directional electrodes configured to receive signals from a particular direction (e.g., from neurons positioned in a particular part of the neuroanatomy), to provide stimulation pulses in a particular direction, such as towards neurons of a particular anatomical structure (e.g., neurons of the DRG, peripheral process, central process, or any of the foregoing), or both. Moreover, one or more of electrodes 108 may be omnidirectional electrodes, such as ring electrodes. It is to be understood, however, that the particular types, geometries, and/or configurations of the one or more electrodes 108 can be adapted based on exigencies of sensing and/or stimulating the neuroanatomy of interest.

Electrodes 108 may be spatially arranged along a lead body of lead 106 according to the target neuroanatomy of a patient, such as to provide electrodes configured to be proximate to the DRG, peripheral process, central process, and/or other neuroanatomy of the patient. For example, electrodes 108 adapted for use in closed loop techniques for treating patient pain via DRG stimulation in accordance with the present disclosure may be grouped in a first plurality of electrodes, a second plurality of electrodes, and a third plurality of electrodes. The first plurality of electrodes may be separated along a length of the lead body from the second plurality of electrodes by a first threshold distance. Additionally, the third plurality of electrodes may be separated along the length of the lead body from the first plurality of electrodes by a second threshold distance.

The first threshold distance, the second threshold distance, or both may be configured to reduce a quantity of noise (e.g., distortions in a neural signal, stimulation artifacts, etc.) observed by one or more of electrodes 108 (e.g., during sensing or recording of neuronal signals). The reduced noise provided via separating the different pluralities of electrodes may enhance a signal to noise (SNR) ratio of electrodes 108 and enhance or improve the quality of neuronal signals recorded during sensing operations. For example, the first threshold distance may reduce noise with respect to signals recorded or sensed by electrodes of the first plurality of electrodes when the sensing is performed simultaneously with or subsequent to stimulation of patient tissue by the second plurality of electrodes. In this manner, feedback data generated by one plurality of electrodes (e.g., the first, second, or third plurality of electrodes) may not be compromised or degraded by noise arising from a stimulation operation performed by an adjacent plurality of electrodes. The second threshold distance between the second plurality of electrodes and the third plurality of electrodes similarly may be configured to mitigate noise and improve the SNR as between the second plurality of electrodes and the third plurality of electrodes. It is noted that while three pluralities of electrodes have been described above, such description has been provided for purposes of illustration, rather than by way of limitation. Thus, it is to be understood that the exemplary closed loop techniques disclosed herein for treating patient pain or other patient conditions may utilize a lead that includes more than three pluralities of electrodes or less than three pluralities of electrodes depending on the particular anatomy of interest, the biomarkers or characteristics to be derived from the feedback data, or other factors.

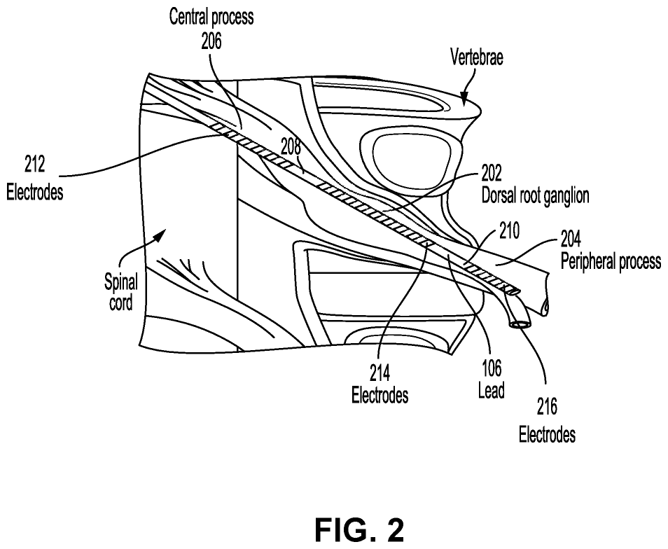

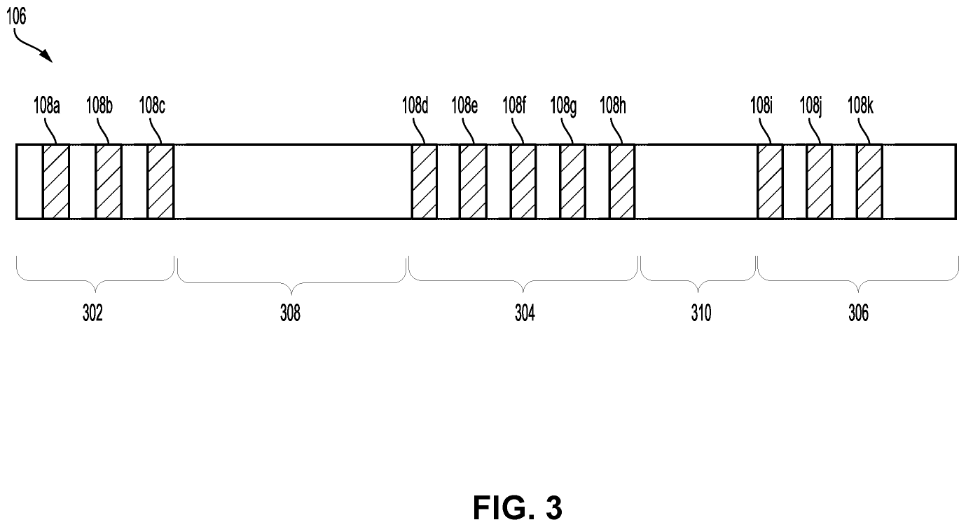

It is noted that different electrode pluralities of electrodes 108 may have different electrode densities. For example, the first plurality of electrodes may include a larger or smaller quantity of electrodes 108 than another plurality of electrodes (e.g., the second plurality of electrodes, the third plurality of electrodes, or both). This may enable regions of high electrode density and/or low electrode density to be defined along a length of lead 106. The ability to define regions of different electrode density along the length of lead 106 may enable customization of lead 106 to specific characteristics of the neuroanatomy of the patient or the needs of a particular stimulation therapy. For example, a lead may be designed to include one or more high density electrode regions and one or more lower density electrode regions distributed along the length of the lead based on the particular therapy and/or neuroanatomy involved. The lead may be implanted in the patient such that the higher density electrode region(s) is positioned proximate neuroanatomy for which a higher degree of control (e.g., for stimulation) and/or a higher sensitivity (e.g., for sensing neuronal activity) are desired while the lower density electrode region(s) is positioned proximate neuroanatomy for which a lower degree of control (e.g., for stimulation) and/or a lower sensitivity (e.g., for sensing neuronal activity) are needed. Exemplary aspects of configuring an arrangement of electrodes on a lead based on the target anatomy and/or therapy are described more fully with reference to FIGS. 2 and 3. Additionally, it is noted that one or more of electrodes 108 may be spatially oriented along lead 106 in a configuration to enhance a SNR of the one or more of electrodes 108 performing sensing operations, to enhance receipt, at neural tissue, of stimulation pulses generated by one or more of electrodes 108 performing stimulation operations, or both. Geometries of one or more of electrodes 108 may also be utilized to facilitate precise control of the induced electric field and, consequently, a volume of neural tissue exposed to the electrical field.

Controller 110 may be a microcontroller having one or more processors, one or more memories, and/or any of the foregoing. The one or more processors may include and/or correspond to one or more microprocessors, central processing units (CPUs), graphical processing units (GPUs), field programmable gate arrays (FPGAs), application specific integrated circuits (ASICs), and/or other logic circuitry configured to perform the operations of controller 110 described herein. Controller 110 may be communicatively coupled to lead 106, to one or more of electrodes 108, to memory 114, and/or to communication interface 118. Controller 110 may include feedback logic 112. Feedback logic 112 may be circuitry, firmware, software, and/or any combination thereof configured to process feedback data received from the one or more of electrodes 108, to analyze the feedback data, and to determine whether to modify one or more stimulation parameters (e.g., an amplitude, a frequency, a pulse width, a polarity, etc.) used to generate stimulation pulses delivered to the target tissue of the patient. Additionally, controller 110 may be configured to control generation of stimulation pulses by the IPG 104 according to the stimulation parameters. In addition to managing generation and delivery of stimulation pulses to select ones of electrodes 108, controller 110 may also be configured to control collection of feedback data corresponding to bioelectrical signals generated by neural tissue by particular ones of electrodes 108. In an aspect, IMD 102 may include one or more switches (not shown in FIG. 1) that may be configured by controller 110 to control delivery of stimulation pulses to the target tissue of the patient by particular ones of electrodes 108 and to control recording or sensing of the feedback data by other ones of electrodes 108. For example, a stimulation switch may be configured to electrically couple different ones of electrodes 108 to the IPG 104 to enable stimulation pulses to be delivered to the target tissue and a sensing switch may be configured to electrically couple one or more of electrodes 108 to enable feedback data to be collected and provided to feedback logic 112 for analysis.

IMD 102 may include memory 114. Memory 114 may include a random access memory (RAM), static RAM (SRAM), dynamic RAM (DRAM), static dynamic RAM (SDRAM), read only memory (ROM), programmable read only member (PROM), erasable programmable read only member (EPROM), electrically erasable programmable read only memory (EEPROM), optical storage, one or more hard disk drives (HDDs), solid state disk drives (SSDs), other memory devices configured to store data, instructions, or both in a persistent or a non-persistent state, or a combination of different memory devices. It is noted that while memory 114 is shown as a standalone component in FIG. 1, memory 114 may be distributed among various components of IMD 102. For example, a portion of memory 114 may be memory devices supporting operations of controller 110 (e.g., cache memory, etc.) or other components of IMD 102.

Memory 114 may store instructions (e.g., software, firmware, etc.) and/or data. For example, memory 114 may include a non-transitory computer-readable storage medium having instructions that, when executed one or more processors (e.g., one or more processors of controller 110), cause the one or more processors to perform operations for providing closed loop stimulation therapy in accordance with aspects of the present disclosure. In an aspect, memory 114 may further be configured to store parameters 116 that include and/or correspond to stimulation parameters used to control operations of IMD 102, such as to control characteristics of the stimulation pulses delivered to the patient (e.g., whether the pulses are continuous or intermittent, a frequency, an amplitude, a pulse width, a polarity, or other parameters). It is noted that the stimulation parameters may be periodically changed or modified, either by operations of patient programmer 120 or clinician programmer 122, based on feedback data received from electrodes 108, or combinations thereof.

In some aspects, parameters 116 may include active parameters (e.g., parameters that may be used to configure stimulation therapies for the patient), as well inactive parameters (e.g., parameters that were previously used to configure stimulation therapies for the patient but which are not being used presently). Storing active and inactive parameter sets at memory 114 may enable a user (e.g., a clinician or the patient) to view historical parameters and current parameters to evaluate aspects of the patient's treatment, such as to identify parameters that were inactivated as being ineffective in treating the patient, active parameters used to effectively treat the patient, or for other purposes. In some aspects, memory 114 may store other types of information, such as data corresponding to one or more biomarkers associated with neuronal signals indicative of a state of the patient's condition (e.g., high pain, moderate pain, and/or low or no pain). Moreover, memory 114 may store data, such as statistical data, electrical data, etc., corresponding to neuronal activity indicative of high pain, moderate pain, and or low or no pain experienced by the patient.

Additionally, IMD 102 may include or be communicatively coupled to other components (not depicted in FIG. 1), such as one or more sensors (e.g., an accelerometer, a gyroscope, a heart rate sensor, a blood pressure sensor, a temperature sensor, galvanic skin conductance sensor, EEG sensor, and/or other types of sensors). As an example, controller 110 may receive sensor data from the one or more sensors, such as accelerometer data from an accelerometer, indicating a mobility state of the patient (e.g., whether the patient is ambulatory, etc.), and the mobility state may provide an additional piece of information that indicates whether the patient is experiencing pain. Controller 110 may analyze the sensor data and the feedback data to determine a state or a condition of the patient (e.g., detecting biomarkers or other indications the patient is experiencing pain) and may adjust the stimulation parameters accordingly.

As briefly noted above, IMD 102 may be communicatively coupled to patient programmer device 120 and/or clinician programmer device 122 via the network(s) 124. To facilitate such communication, IMD 102 may include communication interface 118. Communication interface 118 may be a transceiver, a transmitter, a receiver, or any combination thereof. Additionally or alternatively, communication interface 118 may be networking hardware capable of communicating (e.g., receiving and/or sending data) with external devices using one or more communication standards or protocols (e.g., IEEE 802.11, Bluetooth™, Bluetooth Low Energy (BLE), Zigbee™, any of the 3G, 4G, or 5G communication protocols, other communication protocols, or combinations thereof).

Network 124 may include peer-to-peer networks, wireless fidelity (Wi-Fi) networks, wide area networks (WANs), local area network (LANs), the Internet, or other types of communication networks that may be utilized to facilitate the exchange of data between IMD 102, patient programmer 120, and clinician programmer 122, or combinations thereof. In embodiments, various security measures and protocols (e.g., encryption, certificates, digital signatures, etc.) may be leveraged by system 100 to facilitate secure communication of data exchanged between and among IMD 102, patient programmer 120, and clinician programmer 122 over network 124.

Patient programmer device 120 may be a device (e.g., smartphone, tablet computing device, laptop computing device, or another computing device) configured to provide instructions to and/or receive data from IMD 102. Patient programmer device 120 may be principally associated with the patient in whom IMD 102 is implanted. Clinician programmer 122 may be a device (e.g., smartphone, tablet computing device, laptop computing device, desktop computing device, or other types of computing devices) configured to provide functionality that enables a clinician to create and send instructions to IMD 102 and/or receive data from IMD 102. Clinician programmer 122 may be principally associated with a health care provider. In some aspects, patient programmer 120 and clinician programmer 122 may also be configured to exchange data via network(s) 124.

As briefly described above, IPG 104 may be configured to generate stimulation pulses that may be delivered to target tissue of a patient to treat one or more medical conditions. In an exemplary mode of operation, the stimulation pulses generated by IPG 104 may be delivered to spinal tissue of a patient via one or more electrodes among electrodes 108 to treat patient pain. In this example, the electrodes 108 may be implanted adjacent to the neural tissue of interest (e.g., a central process, a DRG, a peripheral process, etc.). The stimulation pulses may be generated by IPG 104 based on stimulation parameters (e.g., parameters 116) configured to mitigate the pain of the patient. In particular, stimulation parameters used by IPG 104 to generate the stimulation pulses may be configured to block or attenuate transmission or relay of pain signals to the brain.

Delivery of stimulation pulses to target neural tissue of the patient (e.g., the DRG) may block transmission of at least some pain signals, but a one-size-fits all or one-size-fits most approach is insufficient to effectively treat a patient's chronic pain. For example, clinician programmer 122 can be used during a session between the patient and a clinician to configure a set of stimulation parameters that effectively treats a specific level or intensity of pain for the patient, but these static therapy configurations may be insufficient to treat pain experienced by the patient over time. As a result, the patient may experience discomfort (e.g., paresthesia caused by over stimulation of the target tissue) if the stimulation amplitude or frequency is too high, or may experience more intense pain if the pain increases above the level for which the stimulation parameters were intended (e.g., understimulation of the target tissue). Additionally, the pain experienced by the patient may vary according to a mobility state of the patient (e.g., is the patient lying down, sitting, standing, walking, running, and the like). As such, stimulation parameters for a pain management therapy intended for a particular level of pain may become insufficient due to changes in the patient's mobility state. One approach to address the challenges described above is to provide multiple sets of stimulation parameters, each configured for a different level or intensity of pain being experienced by the patient. While providing a greater degree of control over the therapies used to treat the patient's pain, these pre-determined and static configurations still suffer from the same disadvantages described above (e.g., uncomfortable paresthesia due to overstimulation or intense pain due to under-stimulation) since the level of chronic pain experienced by the patient may not be well aligned with any specific one of the preconfigured stimulation parameters.

Closed loop neurostimulation systems offer a more dynamic approach to treating patient pain, but the effectiveness of such systems is limited by the ability to reliably detect biomarkers or other types of triggers that may be used to detect changes in the patient's condition and modify the stimulation parameters accordingly. Treatment of chronic pain using closed loop systems has been challenging due to difficulties identifying biomarkers or other neurological events capable of quantifying patient pain, as well as challenges with respect to reliable detection of signals that may potentially serve as biomarkers suitable for closed loop pain management systems. Below, exemplary aspects of closed loop systems and methods capable of addressing the above-described challenges are described.

As briefly described above, IMD 102 may be operated in a closed manner to treat chronic pain of a patient. In particular, controller 110 may be configured to receive feedback data from particular electrodes of electrodes 108. The feedback data may correspond to neuronal signals (e.g., bioelectrical signals) indicative of pain (e.g., pain signals) as well as neuronal signals generated in response to delivery of the one or more stimulation pulses to the target tissue of the patient. Exemplary types of neuronal signals that may be received as feedback data by controller 110 may include electroneurogram data corresponding to pain signals generated at neural tissue of the patient, ECAP data, and/or neuronal data corresponding to neuronal activity of particular neurons of the neuroanatomy of the patient, such as neuronal activity of nociceptive neurons of a DRG of the patient. As described in more detail below, the above-described neuronal signals may be used, individually or in combination, as biomarkers for evaluating the pain state of a patient and may provide a basis for quantifying a patient's pain in a manner that enables stimulation parameters to be adjusted in a closed loop manner to dynamically maintain patient pain at comfortable levels.

Controller 110 may be configured to determine whether to modify the stimulation parameters based, at least in part, on the feedback data. For example, a pain level of the patient can be estimated by recording neuronal activity with the electrode contacts placed on or near the DRG, since chronic neuropathic pain can be characterized by hyperactivity of the nociceptive neurons. In addition, ENG recordings with electrode contacts placed on or near the dorsal root or rootlets can be incorporated to estimate the patient's pain level. To further illustrate, feedback logic 112 of controller 110 may be configured to receive feedback data as an input and to determine, based on the feedback data, whether to modify one or more stimulation parameters. In some aspects, the adjustment of the stimulation parameters may be configured to enhance a blocking effect of stimulation pulses delivered to the DRG. For example, pain signals originating in peripheral nerves or tissue may enter the DRG via the peripheral process of the pseudo-unipolar nociceptive neuron and then propagate to the central process and then on to the spinal cord and the brain, at which point the patient perceives the pain indicated by those pain signals. However, stimulation pulses delivered to the DRG via electrodes 108 may block all or a portion of those pain signals and prevent the pain signals from propagating to the central process, thereby preventing the pain signals (or a portion thereof) from reaching the patient's brain thus reducing the perceived pain of the patient. The term “blocking effect” may refer to how delivery of stimulation pulses to the DRG attenuates or prevents transmission of pain signals from the DRG to other neuroanatomy of the patient, such as transmission of pain signals from the DRG to the central process of the patient. An extent to which the blocking effect attenuates propagation of the pain signals to the central process may serve as a biomarker for evaluating a pain state of the patient suitable for providing closed loop control of neurostimulation therapies to treat chronic pain.

Additionally or alternatively, the blocking effect may correspond to the effectiveness of stimulation pulses delivered to other anatomy of the patient (e.g., besides the DRG) at inducing an inhibitory response in neurons resulting in attenuation of pain signal generation and/or transmission. For example, stimulation pulses delivered to the peripheral process may be used either to block pain signals originating in peripheral nerves or tissue from entering the peripheral process or to activate other sensory modality signals, such as recruiting the somatosensory fibers to mitigate pain. As described in more detail below, system 100 may include various features that enable reliable detection of neuronal signals for detection of blocking-effect type biomarkers for use in a closed loop system for treating chronic patient pain.

As briefly described above, feedback logic 112 of controller 110 may provide functionality for determining whether to modify or adjust stimulation parameters based on the feedback data. The functionality provided by feedback logic 112 may be configured to analyze the feedback data, which may include detecting the presence of a biomarker indicative of patient pain, such as the blocking effect described above, and modify or adjust the stimulation parameters when a biomarker is detected. In an aspect, modification of the stimulation parameters may be configured to improve mitigation of patient pain, such as to enhance or improve a blocking effect resulting from delivery of the stimulation pulses.

In response to a determination to adjust the stimulation parameters, controller 110 (or feedback logic 112) may be configured to modify one or more of the stimulation parameters to produce a modified set of stimulation parameters. For instance, an amplitude of one or more stimulation pulses delivered to the DRG may be configured to block an action potential propagation at the T-junction of nociceptive neurons. The stimulation pulse amplitudes are increased when the stimulation induced pain blocking effect with the aforementioned methods is below the desired level, and the stimulation pulse amplitudes can be decreased or the stimulation can be turned off temporarily to enhance the battery longevity while monitoring the pain blocking effect aforementioned. Other stimulation parameters can be also adjusted to maximize the therapeutic efficacy of neurostimulation including stimulation frequency and/or the temporal patterns of the stimulation waveforms. The modified set of stimulation parameters may be configured to enhance mitigation of the pain of the patient. In particular, the modified set of stimulation parameters may be configured to enhance a blocking effect achieved via delivery of the stimulation pulses to target tissue of the patient. The enhanced blocking effect may improve attenuation of the pain signals, resulting in less pain signals propagating to the patient's brain so that the patient experiences or senses a lower level of pain than the patient would otherwise experience. Exemplary stimulation parameters that may be modified or adjusted include frequency, amplitude, pulse width, electrode configuration (e.g., selection of the anodes and cathodes used to deliver stimulation pulses), burst pattern, and the like. To enhance the therapeutic benefit of electrical stimulation and reduce undesired side effects, including stimulation induced discomfort, these parameters may be limited within certain boundary values and closed loop control techniques facilitate the automatic adjustment of these stimulation parameters within the configured boundary values. For example, a clinician may use a clinician programmer device to configure the boundary values (e.g., an upper and lower threshold for different stimulation parameters, such as frequency, amplitude, pulse width, and the like) during a programming session initiated between an IMD and the clinician programming device. Once the boundary values are configured, adjustment of parameters in accordance with the closed loop techniques disclosed herein may be limited to parameter values within the boundary values configured by the clinician. It is noted that in some instances the modification of the stimulation parameters may be configured to reduce the blocking effect. For example, discomfort may be experienced by the patient when target tissue of the patient is overstimulated. This may occur when the stimulation parameters are configured for high levels of pain and the patient is experiencing low levels of pain. In such instances, controller 110 may modify the stimulation parameters to reduce the blocking effect to prevent the patient from experiencing uncomfortable paresthesia. When modifying the stimulation parameters to lower the blocking effect, controller 110 may ramp down the stimulation parameters gradually until a desired blocking effect is achieved. In this manner, the discomfort caused by overstimulation may be mitigated without unintentionally dropping the effectiveness of the blocking effect to a level that is too low and causes the patient's perceived pain level to increase sharply.

Additional stimulation pulses configured based on the modified set of stimulation parameters may be subsequently delivered to the spinal tissue of the patient via at least one electrode of the plurality of electrodes 108. For instance, controller 110 may cause IPG 104 to generate stimulation pulses according to the modified set of stimulation parameters, and the stimulation pulses generated based on the modified set of stimulation parameters may be delivered to DRG of the patient, other neural tissue of the patient, or both via the at least one electrode of the plurality of electrodes 108. It is noted that in some instances controller 110 (or feedback logic 112) may determine, based on the feedback data, not to modify the stimulation parameters (e.g., because the stimulation pulses are adequately mitigating the patient's perceived pain levels). In such instances, controller 110 may nevertheless cause IPG 104 to deliver additional stimulation pulses to the neural tissue of the patient using the unmodified set of stimulation parameters in order to maintain mitigation of patient pain at a consistent level. In some aspects, if controller 110 determines not to modify the stimulation parameters based, at least in part, on the feedback data, controller 110 may be configured to delay further delivery of stimulation pulses until additional feedback data is received. Delaying delivery of the additional stimulation pulses in this manner may enable controller 110 to evaluate whether patient pain has subsided, in which case no further stimulation is needed, or remains present, at which point stimulation may resume as described above.

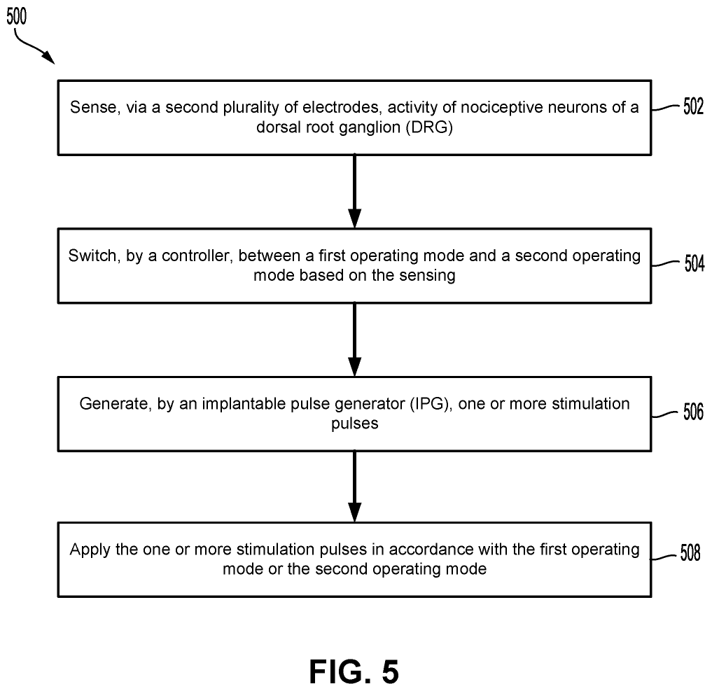

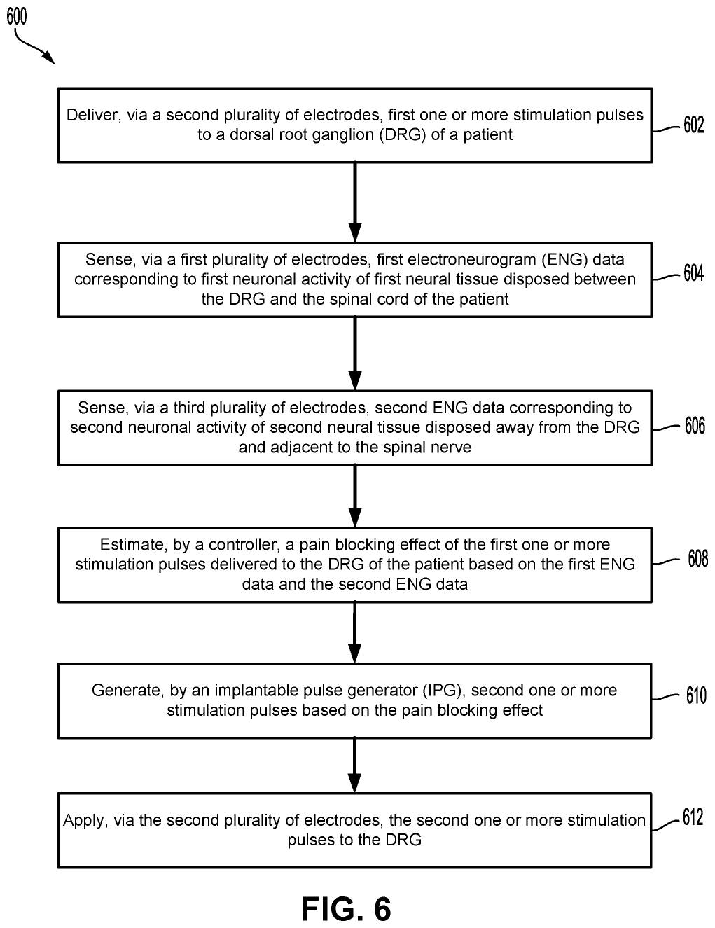

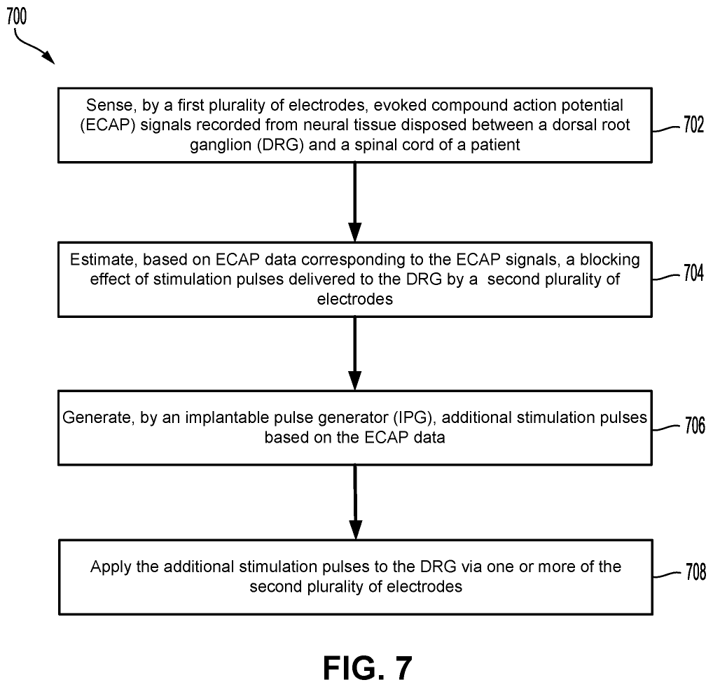

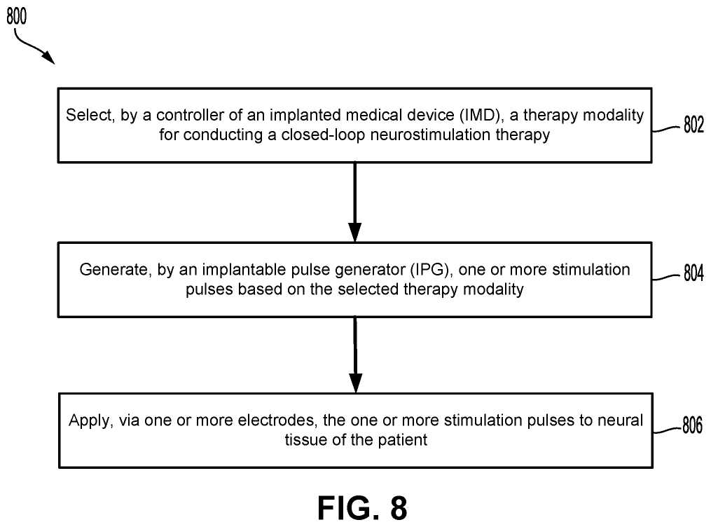

Closed loop electrical stimulation techniques in accordance with embodiments of the present disclosure may leverage different approaches with respect to evaluating the patient pain state, detecting the presence of biomarkers indicative of pain, and enhancing an effectiveness of stimulation pulses for pain mitigation. For example, the feedback data may include neuronal data corresponding to neuronal activity of particular neural clusters in the neuroanatomy of the patient, such as neuronal activity of nociceptive neurons of the DRG of the patient. Exemplary techniques for using feedback data that include neuronal activity of nociceptive neurons in the DRG to manage chronic pain in a closed loop manner are described in more detail below with reference to FIG. 5. As another example, the feedback data may include ENG data simultaneously collected or recorded from the central process and the peripheral process during stimulation of the DRG. Exemplary techniques for using feedback data that includes ENG data to manage chronic pain in a closed loop manner are described in more detail below with reference to FIG. 6. In yet another example, the feedback data may include ECAP data. Exemplary techniques for using feedback data that includes ECAP data to manage chronic pain in a closed loop manner are described in more detail below with reference to FIG. 7. Moreover, exemplary closed loop electrical stimulation techniques using combinations of neuronal activity of nociceptive neurons, ENG data, and ECAP data are described in more detail below with reference to FIG. 8.

Leveraging the above-described functionality enables system 100 to monitor and treat patient pain in a closed loop manner. To reliably provide closed loop control, system 100 incorporates a lead having electrodes configured to facilitate both stimulation of target neural tissue of the patient and recording of feedback data that may provide reliable biomarkers of the patient's pain state. The electrodes utilized by system 100 may be designed in a manner that mitigates noise and enhances the SNR of the electrodes utilized for sensing, while also providing a higher degree of directionality and control with respect to both sensing and delivery of stimulation pulses. Exemplary aspects of electrode configurations providing improved SNR and noise mitigation, as well as directional control are described below with reference to FIGS. 2 and 3. The enhanced capabilities of the electrodes utilized by system 100 enable reliable feedback to be recorded. The recorded feedback data may be analyzed by system 100 and used to determine whether to adjust one or more of parameters 116 (e.g., stimulation parameters). Modification of the one or more parameters 116 may be performed in certain situations based on the analysis of the feedback data, such as when modification of the one or more parameters will enhance an efficacy of a therapy for treating pain of the patient. As shown above, the improved SNR and reduced noise provided by the configuration of the electrodes utilized by system 100 to sense or record the feedback data may enable various signals and biomarkers indicative of patient pain to be reliably monitored by system 100. The ability to reliably observe the signals and biomarkers provides a framework that may be used by system 100 to control a stimulation therapy to manage chronic pain of a patient in a closed loop manner.

Referring to FIG. 2, an image illustrating aspects of a lead placed within spinal tissue of a patient according to embodiments of the present disclosure is depicted. As described above with reference to FIG. 1, lead 106 includes a plurality of electrodes 108. In FIG. 2, the electrodes 108 of lead 106 are shown to include a first plurality of electrodes 212, a second plurality of electrodes 214, and a third plurality of electrodes 216. In the exemplary arrangement shown in FIG. 2, electrodes 108 are disposed along the length of lead 106 such that first plurality of electrodes 212 is configured to be disposed between DRG 202 and a spinal cord of the patient and adjacent to central process 206 of the patient when the lead body of lead 106 is inserted into the patient such that first plurality of electrodes 212 are proximate to the central process of pseudo-unipolar (nociceptive and somatosensory) neurons. Additionally, second plurality of electrodes 214 is configured to be disposed adjacent to DRG 202 of the patient when the lead body of lead 106 is inserted into the patient, and third plurality of electrodes 216 is configured to be disposed away from the DRG 202 and adjacent to a spinal nerve from which the DRG 202 emerges when the lead body of lead 106 is inserted into the patient such that third plurality of electrodes 216 is proximate to the peripheral process of pseudo-unipolar (nociceptive and somatosensory) neurons (e.g., a peripheral process 204).

In the arrangement depicted in FIG. 2, second plurality of electrodes 214 is positioned between first plurality of electrodes 212 and third plurality electrodes 216. Moreover, second plurality of electrodes 214 is separated from first plurality of electrodes 212 along the length of the lead body of lead 106 by first threshold distance 208. Third plurality of electrodes 216 is separated from second plurality of electrodes 214 by second threshold distance 210 along the length of the lead body of lead 106. First threshold distance 208 is configured to mitigate noise and interference between second plurality of electrodes 214 and first plurality of electrodes 212, such as noise or stimulation artifacts that may arise during sensing of neuronal signals or interference during stimulation (e.g., prevent stimulation provided via second plurality of electrodes 214 from bleeding over to central process 206 or stimulation provided via first plurality of electrodes 212 from bleeding over to DRG 202). Similarly, second threshold distance 210 is configured to mitigate noise and interference between second plurality of electrodes 214 and third plurality of electrodes 216, such as noise or stimulation artifacts that may arise during sensing of the neuronal signals or interference during stimulation (e.g., prevent stimulation provided via second plurality of electrodes 214 from bleeding over to the peripheral process 204 or stimulation provided via third plurality of electrodes 216 from bleeding over to DRG 202). To illustrate, if stimulation pulses are applied to peripheral process 204 via third plurality of electrodes 216 and electrodes of second plurality of electrodes 214 proximate DRG 202 are configured to sense neuronal signals during or soon after delivery of the stimulation pulses to peripheral process 204, second threshold distance 210 is configured to mitigate noise and stimulation artifacts received at electrodes of second plurality of electrodes 212 proximate to DRG 202 and possibly resulting from delivery of the stimulation pulses to peripheral process 204.

As explained with reference to FIG. 1, electrode density may correspond to a quantity and/or relative spacing of electrodes 108 along the lead body of lead 106. For instance, second plurality of electrodes 214 may have a first electrode density based on a number of electrodes disposed along a length of the lead body of lead 106 corresponding to second plurality of electrodes 214; first plurality of electrodes 212 may have a second electrode density based on a number of electrodes disposed along a length of the lead body of lead 106 corresponding to second plurality of electrodes 214; and third plurality of electrodes 216 may have a third electrode density based on a number of electrodes disposed along a length of the lead body of lead 106 corresponding to third plurality of electrodes 216. In some aspects, the first electrode density may be greater than, equal to, or less than the second electrode density, the third electrode density, or both. In additional or alternative aspects, the second electrode density may be greater than, equal to, or less than the first electrode density, third electrode density, or both. In additional or alternative aspects, the third electrode density may be greater than, equal to, or less than the first electrode density, second electrode density, or both.

Incorporating different electrode density regions along the length of the lead body of lead 106, as shown in FIG. 2, and separating each of the different regions by a threshold distance (e.g., the above-described first and second threshold distances) enables sensing to be performed more efficiently (e.g., due to increased SNR and mitigated noise between different ones of the pluralities of electrodes). Moreover, configuring lead 106 to include different electrode regions as shown in FIG. 2 (e.g., with electrode pluralities configured to be positioned proximate the peripheral process, the DRG, and the central process when lead 106 is implanted in a patient) enables the above-described feedback data and biomarkers to be reliably recorded and identified, thereby enabling utilization of closed loop stimulation techniques to reliably treat patient pain. It is noted that while utilization of different electrode regions may be advantageous for the reasons explained above, other aspects of the configuration of the electrodes of lead 106 may provide additional advantages. For example, an orientation or size of the electrodes may be used to control delivery of stimulation to precise regions of the target tissue (e.g., the DRG, peripheral process, central process, or other tissue) of the patient, and may also enable sensing of neuronal signals from particular portions of the target tissue (e.g., the DRG, peripheral process, central process, or other tissue). This may enhance control of an electric field generated by one or more of electrodes of lead 106 during delivery of a stimulation pulse to the target tissue and may also help mitigate stimulation of undesired tissue of the patient. Exemplary aspects of electrode configurations providing increased control for delivery of stimulation pulses and recording feedback data suitable for identification of biomarkers enabling closed loop stimulation in accordance with aspects of the present disclosure are described in more detail below with reference to FIGS. 3 and 9A-10B.

Referring to FIG. 3, a block diagram illustrating a lead for performing closed loop stimulation therapy to treat pain of a patient according to embodiments of the present disclosure is presented. In FIG. 3, lead 106 of FIG. 1 is shown to include second plurality of electrodes 304 that include electrodes 108d-108h, first plurality of electrodes 302 that include electrodes 108a-108c, and third plurality of electrodes 306 that include electrodes 108i-108k. In an aspect, second plurality of electrodes 304 may be second plurality of electrodes 214 of FIG. 2, first plurality of electrodes 302 may be first plurality of electrodes 212 of FIG. 2, and third plurality of electrodes 306 may be third plurality of electrodes 216 of FIG. 2. As shown in FIG. 3, first plurality of electrodes 302 is positioned along the length of lead 106 on a first side of second plurality of electrodes 304 and third plurality of electrodes 306 positioned along the length of lead 106 on a second side of second plurality of electrodes 304 (i.e., second plurality of electrodes 304 is between first plurality of electrodes 302 and third plurality of electrodes 306). Additionally, second plurality of electrodes 304 is separated from first plurality of electrodes 302 along a length of a lead body of lead 106 by first threshold distance 308. Second plurality of electrodes 304 is also separated from third plurality of electrodes 306 along the length of lead body of lead 106 by second threshold distance 310. First threshold distance 308 may be equal to, greater than, or less than second threshold distance 310.

As explained with reference to FIGS. 1 and 2, first threshold distance 308 and second threshold distance 310 may be configured to reduce noise with respect to signals sensed by one or more of electrodes 108a-108k during a stimulation operation. To illustrate, while electrode 108f delivers one or more stimulation pulses to one or more neurons, electrode 108c and electrode 108i may record neuronal data corresponding to signals generated by one or more neurons proximate to electrodes 108c, 108i, respectively. First threshold distance 308 and second threshold distance 310 separating electrode 108f from electrodes 108c and 108i may be configured to reduce noise sensed at electrodes 108c, 108i from the stimulation pulses delivered to tissue of the patient by electrode 108f, thereby enhancing a SNR of the feedback data corresponding to the or recorded signals sensed by electrodes 108c and 108i. Additionally, the separation between the different pluralities of electrodes 302-306 may also minimize or mitigate interference with stimulation pulses delivered by different electrodes of the pluralities of electrodes 302-306. For example, stimulation pulses delivered by first plurality of electrodes 302 (e.g., one or more of electrodes 108a-108c) may be isolated to tissue proximate second plurality of electrodes 304 and may not be delivered to or bleed over to tissue proximate first plurality of electrodes 302 or third plurality of electrodes 306. Thus, when first plurality of electrodes 302 and/or third plurality of electrodes 306 deliver stimulation pulses to tissue of the patient simultaneously with delivery of stimulation pulses by electrodes of second plurality of electrodes 304, the impact of the stimulation pulses delivered to tissue of the patient by each different plurality of electrodes may be limited to target tissue proximate each respective plurality of electrodes (i.e., stimulation by one plurality of electrodes will not bleed over to tissue proximate another plurality of electrodes and interfere with stimulation pulses or recording of feedback data by the other plurality of electrodes). It is understood that different geometries and configurations of electrodes may be used with leads to provide closed loop control of neurostimulation therapies to treat pain in accordance with the concepts described herein. The electrode geometries and configurations along a lead, such as lead 106, may be differently configured, shaped, and oriented to enhance a SNR of signals received from one or more neurons, to more precisely and accurately steer current to neural tissue of the patient, or both.

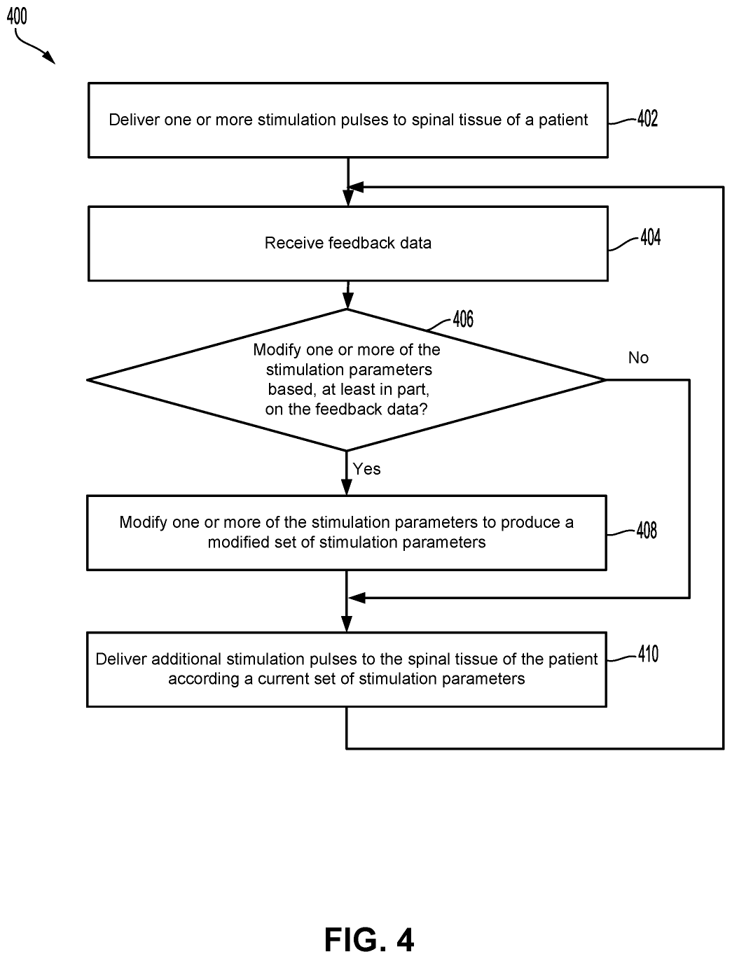

Referring to FIG. 4, a flowchart depicting an exemplary method for mitigating pain of a patient using closed loop electrical stimulation according to embodiments of the present disclosure is shown as a method 400. At block 402, method 400 includes delivering one or more stimulation pulses (e.g., stimulation pulses generated by an IPG, such as IPG 104 of FIG. 1) to spinal tissue of a patient via first one or more electrodes (e.g., by one or more of electrodes 108 of FIGS. 1-3). The first one or more electrodes used to deliver the one or more stimulation pulses may include electrodes selected from a second plurality of electrodes (e.g., second plurality of electrodes 214 of FIG. 2 or 304 of FIG. 3), a first plurality of electrodes (e.g., first plurality of electrodes 212 of FIG. 2 or 302 of FIG. 3), and a third plurality of electrodes (e.g., third plurality of electrodes 216 of FIG. 2 or 306 of FIG. 3). At block 404, method 400 includes receiving feedback data from second one or more electrodes (e.g., by one or more of electrodes 108 of FIG. 1). The second one or more electrodes may include electrodes selected from a second plurality of electrodes (e.g., second plurality of electrodes 214 of FIG. 2 or 304 of FIG. 3), a first plurality of electrodes (e.g., first plurality of electrodes 212 of FIG. 2 or 302 of FIG. 3), and a third plurality of electrodes (e.g., third plurality of electrodes 216 of FIG. 2 or 306 of FIG. 3). Exemplary aspects of controlling delivery of stimulation pulses to target tissue of the patient and obtaining feedback data from the target tissue (or tissue proximate the target tissue) using different sets of electrodes are described in more detail below with reference to FIGS. 5-7.

At block 406, method 400 includes determining whether to modify one or more stimulation parameters (e.g., stimulation parameters used to generate the one or more stimulation pulses delivered at 402) based, at least in part, on the feedback data. As described herein, the modification of the stimulation parameters may be configured to enhance or improve an efficacy of the stimulation pulses, such as to mitigate pain of the patient without the discomfort caused by overstimulation or the loss of the therapeutic efficacy due to understimulation (e.g., common problems of prior stimulation systems for treating pain of a patient). It is noted that the determination to modify (or not modify) the stimulation parameters and the particular feedback considered, at block 406, may depend on the biomarker(s) utilized to identify a pain state or level of the patient. Exemplary techniques for determining whether to modify the one or more stimulation pulses based on feedback data and one or more biomarkers of interest are described in more detail below with reference to FIGS. 5-9. At block 408, method 400 includes modifying the one or more stimulation parameters to produce a modified set of stimulation parameters in response to determining to modify the one or more stimulation parameters. It is noted that in some aspects block 408 may instead determine not to modify the stimulation parameters (e.g., if the feedback data and biomarkers of interest indicate that the patient's pain state or level is adequately being treated by a current set of stimulation parameters).

At block 410, method 400 includes delivering additional stimulation pulses to the spinal tissue of the patient via at least one electrode of the plurality of electrodes according to a current set of stimulation parameters. It is noted that the current set of stimulation parameters may include the modified set of stimulation parameters or the set of stimulation parameters used to generate the stimulation pulses delivered at block 402 depending on whether the determining, at block 406, indicates the patient's pain is adequately being treated or inadequately being treated. For example, if, at block 406, it is determined not to modify the one or more of the stimulation parameters, method 400 may proceed to block 410 and additional stimulation pulses may be delivered to the neural tissue of the patient via the first one or more electrodes. After block 410, process 400 may return to 404 at which feedback data again may be received from one or more electrodes.

To elaborate and placing process 400 in the context of FIGS. 1-3, at block 402, a controller (e.g., controller 110 of FIG. 1) of an IMD (e.g., IMD 102) may be configured to cause an IPG (e.g., IPG 104 of FIG. 1) to generate one or more stimulation pulses according to stimulation parameters (e.g., parameters 116 of FIG. 1). The stimulation pulses may be delivered to neural tissue of a patient via one or more electrodes (e.g., electrodes 108 of FIG. 1-3). At block 404, the controller may be configured to receive feedback data. As described herein, the feedback data may be received from second one or more electrodes, which may be the same as or different from the first one or more electrodes used to deliver the one or more stimulation pulses at block 402. The feedback data may correspond to neuronal signals generated in response to being stimulated by the stimulation pulses before, during, or after stimulation. Alternatively or additionally, the feedback data may correspond to neuronal signals collected from neural tissue that has not been exposed to stimulation pulses, such as neuronal signals associated with patient pain.

At block 406, feedback logic (e.g., feedback logic 112 of FIG. 1) of the controller may be configured to analyze the feedback data to determine whether to modify the one or more stimulation parameters. For instance, the feedback logic may be configured to assess an effectiveness of the stimulation pulses in blocking generation and/or transmission of pain signals at nerve tissue of the patient based, at least in part, on the feedback data. Exemplary techniques are described below for analyzing feedback data to assess the effectiveness of the stimulation pulses with respect to blocking the generation and/or transmission of pain signals at nerve tissue of the patient. At block 408, in response to determining to modify one or more of the stimulation parameters, the controller may be configured to modify the one or more stimulation parameters to produce a modified set of stimulation parameters. The modified set of stimulation parameters may be configured to enhance an effectiveness of stimulation pulses in attenuating generation and/or transmission of pain signals emanating from neural tissue of the patient, such as attenuating signals transmitted from the DRG toward the dorsal root or rootlets of the patient. Thereafter, at block 410, the controller may be configured to deliver additional stimulation pulses to the spinal tissue (or other neural tissue) of the patient via at least one electrode of the plurality of electrodes according to the modified set of stimulation parameters. Thenceforth, the controller may be configured to cause feedback data to again be received from second one or more electrodes for the purpose of evaluating an effectiveness of the modified set of stimulation parameters at blocking pain signal generation and/or propagation (e.g., minimizing or mitigating the patient's perception of pain).

However, if, at block 406, the controller determines not to modify the one or more of the stimulation parameters, the controller may be configured to cause an IPG to deliver additional stimulation pulses to the spinal tissue of the patient via at least one electrode of the plurality of electrodes according to the unmodified stimulation parameters. Thereafter, the controller may be configured to cause feedback data to again be received from second one or more electrodes for the purpose of re-evaluating an effectiveness of the stimulation parameters at blocking pain signal generation and/or propagation. Alternatively, in lieu of causing an IPG to deliver additional stimulation pulses to the neural tissue of the patient, the controller may be configured to temporarily cease delivery of additional stimulation pulses unless feedback data is received at the controller indicating that the patient is in pain. For example, in response to receipt of feedback data indicating heightened neural activity of neurons involved in propagation of pain signals, the controller may be configured to resume delivery of stimulation pulses.

As shown above, FIG. 4 provides an overview of a closed loop stimulation algorithm for treating pain of a patient in accordance with aspects of the present disclosure. Method 400 and leads constructed according to aspects of the present disclosure may be particularly well suited for applications involving certain neurological areas, such as the DRG. In particular, utilizing a lead having multiple spaced apart sets of electrodes may enable stimulating and sensing operations to be conducted sequentially or simultaneously in a manner that minimizes the impact of the stimulation on sensing operations (e.g., minimizing stimulation artefacts, improving signal quality of sensed feedback, etc.) and vice versa. As a result, neurostimulation systems operating in accordance with the present disclosure, whether according to the method 400 or one of the other methods disclosed herein, may provide improved capabilities to capture feedback for use as inputs to closed loop stimulation algorithms and control processes and provide improved closed loop control of stimulation therapies utilized to treat patient pain. It should be understood that the closed loop stimulation algorithms disclosed herein are not limited to method 400. Additional aspects of closed loop stimulation algorithms and techniques for identifying biomarkers of pain based on feedback data and determining whether to modify stimulation parameters based on the biomarkers and feedback data are described in more detail below with reference to FIGS. 5-8.

Referring to FIG. 5, a flowchart depicting an exemplary method for managing patient pain via closed loop stimulation according to embodiments of the present disclosure is shown as method 500. In an aspect, method 500 may be performed by one or more components of a neurostimulation system, such as IMD 102 of system 100 of FIG. 1. Steps of method 500 may be stored as instructions in a memory of the IMD (e.g., the memory 114 of FIG. 1). The instructions may be executable by one or more processors (e.g., processors of the IMD 102, the controller 114 of FIG. 1, or other logic) to perform closed loop stimulation in connection with managing a patient's pain in accordance with the concepts disclosed herein.

At block 502, method 500 includes sensing activity of nociceptive neurons of the DRG. The activity of the nociceptive neurons may be indicative of neuropathic pain of the patient. At block 504, method 500 includes switching an operating mode of the neurostimulation system (e.g., system 100 of FIG. 1) between a first operating mode and a second operating mode based on the sensing performed at block 502. The first operating mode includes sensing the activity of the nociceptive neurons at least partially simultaneously with delivery of one or more stimulation pulses to the patient (e.g., to the DRG of the patient). The second operating mode includes performing the sensing (e.g. the sensing operation of block 502) in between delivery of the one or more stimulation pulses to the patient. At block 506, one or more stimulation pulses may be generated using the IPG, such as IPG 104 of FIG. 1. At block 508, one or more stimulation pulses may be applied (e.g., to the DRG of the patient by one or more electrodes of the second plurality of electrodes 214, 304 of FIGS. 2 and 3 of an IMD, such as IMD 102 of FIG. 1) in accordance with the first operating mode and the second operating mode.

In an example of method 500 and referring to FIGS. 1-3, at block 502, one or more electrodes of second plurality of electrodes 214, 304 of FIGS. 2 and 3 of an IMD, such as IMD 102 of FIG. 1, may be configured to perform the sensing operation(s) at the DRG to sense the activity of the nociceptive neurons of the DRG. A controller, such as controller 110 of IMD 102 of FIG. 1, may receive feedback data that includes neuronal data received through sensing the activity of neuron clusters, such as the activity of the nociceptive neurons. The feedback data from the DRG, where the cell bodies of the nociceptive neurons reside, may include ambient noise and stimulation artifacts when a sensing operation occurs simultaneously with stimulation, such as if one or more electrodes of second plurality of electrodes 214, 304 of FIGS. 2 and 3 perform a sensing operation while other of second plurality of electrodes 214, 304 of FIGS. 2 and 3 deliver stimulation pulses to the DRG (or electrodes of first plurality of electrodes 212, 302 of FIGS. 2 and 3 or third plurality of electrodes 216, 306 of FIGS. 2 and 3 deliver the stimulation pulses). Therefore, it may be desirable to assess the noise level to avoid modifying stimulation parameter based on signal changes (e.g., in the feedback data) caused by noise.

For instance, controller 110 of IMD 102 of FIG. 1 may receive the feedback data via the first one or more electrodes of second plurality of electrodes 214, 304 of FIGS. 2 and 3. Controller 110 may be configured to determine one or more metrics based on the feedback data. The one or more metrics may include a neuronal activity level metric and a noise metric. The neuronal activity level metric may be indicative of the activity level of neuron clusters, such as of the nociceptive neurons of the DRG. The activity level of nociceptive neurons of the DRG may vary depending on the pain level that a patient perceives. The noise metric may be indicative of interference (e.g., stimulation artifacts, etc.) caused by delivery of one or more stimulation pulses to neural tissue of the patient. Method 500 may determine to switch the operating mode of IMD 102 based on whether the noise level satisfies a noise level threshold. The noise level threshold may correspond to an amount of stimulation artifacts sufficient to create difficulties in detecting changes in the neuronal activity level. Modifying the stimulation parameters may be performed to control whether the neuronal activity is recorded simultaneously with or in between delivery of stimulation pulses or pulse trains, such as to switch between simultaneous and sequential recording of the neuronal data based on the noise level obtained from the feedback data. When the noise level is low (e.g., the noise level is below or does not satisfy the noise level threshold), neuronal activity may be more accurately and readily recorded, enabling simultaneous recording of the neuronal activity while delivering stimulation pulses. When neuronal activity is recorded while stimulation is being delivered, stimulation artifact removal processes (e.g., filtering, etc.) may be incorporated to extract pain signals related to neuronal activity from the sensed neuronal activity. However, when the noise level satisfies the noise level threshold, noise may be high and recording of neuronal data may be switched to sequential mode (i.e., neuronal activity data may be captured when stimulation pulses are not being delivered to tissue of the patient), thereby enabling the impact of the noise on the feedback data to be minimized and enabling adjustments to the stimulation parameters to more accurately reflect a current state of the neurological system, which may enable the patient's pain to be mitigated more effectively than if the noise was present.

Controller 110 may be configured to determine whether to switch the operating mode between the first operating mode and the second operating mode based on whether the noise metric satisfies the noise level threshold (e.g., a value stored in memory 114 of FIG. 1). To illustrate, the noise level threshold may correspond to an amount of stimulation artifacts (or other types of noise) sufficient to create difficulties in detecting changes in the neuronal activity level or other sensed signals used as feedback to characterize and evaluate the effectiveness of neurostimulation with respect to treating the patient's perceived pain. Controller 110 may use the noise metric to determine whether IMD 102 is to be operated in a first operating mode where neuronal activity is recorded (or sensed) simultaneously with delivery of stimulation or in a second operating mode where neuronal activity is recorded (or sensed) in between delivery of stimulation. When the noise level metric does not satisfy the second threshold level (e.g., the noise impact on recorded or sensed neuronal activity is low), IMD 102 may be configured to operate in the first operating mode, enabling simultaneous recording of the neuronal activity while delivering stimulation pulses (e.g., since neuronal activity may be more accurately and readily recorded due to the low noise level). However, when the noise level metric satisfies the noise level threshold, IMD 102 may be configured to operate in the second operating mode where neuronal activity data is captured when stimulation pulses are not being delivered to tissue of the patient (e.g., to minimize the impact of noise on recording of the neuronal activity).

It is noted that in addition to determining whether to configure IMD 102 in the first operating mode (e.g., simultaneous stimulation and sensing) or the second operating mode (e.g., sequential stimulation and sensing), controller 110 may also be configured to determine whether to modify at least one stimulation parameter of one or more stimulation parameters (e.g., parameters 116 of FIG. 1) based on the sensed neuronal activity. For example, when a neuronal activity level metric satisfies a first threshold value (e.g., a value indicative of hyperactivity) or a sudden spike or change in activity level of neuron clusters (e.g., such as nociceptive neurons) is detected, such signals may indicate that the patient is experiencing pain. The one or more stimulation parameters may be modified (e.g., by controller 110) based on the sensed neuronal activity to reduce the patient's perceived pain. For instance, controller 110 may be configured to modify at least one stimulation parameter of the one or more stimulation parameters in response to determining that the neuronal activity level metric satisfies the first threshold level, and the one or more stimulation pulses may be generated (e.g., at IPG 104) based on the least one modified stimulation parameter (e.g., at block 506). The first threshold level may be stored in memory 114 of FIG. 1, and the one or more stimulation parameters may correspond to any of an amplitude of the one or more stimulation pulses, a polarity of the one or more stimulation pulses, a modulation of the one or more stimulation pulses, timing parameters, or other stimulation parameters, as explained in more detail above with respect to FIG. 1. It is noted that the noise level threshold, the first threshold level, or other parameters or thresholds used to control the operating mode and/or characteristics of and adjustments to the stimulation pulses may be stored in a memory (e.g., memory 114 of FIG. 1) and may be updated continuously or periodically to account for the physiological changes of the surrounding tissue, electrode impedance (e.g., due to tissue encapsulation, etc.), lead migration, or other factors.

It is noted that method 500 may additionally sense information associated with a state of the patient via one or more sensors disposed along lead 106 of FIG. 1, elsewhere in the neurostimulation system (e.g., system 100 of FIG. 1 such as disposed in IMD 102), or both. For instance, the one or more sensors may include an accelerometer, a gyroscope, or other types of sensors configured to identify whether the patient is stationary, standing, walking, running, and the like. Controller 110 may receive feedback data from the one or more sensors that includes state data corresponding to a state of the patient (e.g., whether the patient is ambulatory, seated, etc.) and may modify one or more stimulation parameters (e.g., parameters 116 of FIG. 1) based on the state of the patient. One or more stimulation pulses may be generated (e.g., at IPG 104) based on one or more modified stimulation parameters.

Referring to FIG. 6, a flowchart depicting another exemplary method for managing patient pain via closed loop stimulation according to embodiments of the present disclosure is shown as method 600. In an aspect, method 600 may be performed by one or more components of a neurostimulation system (e.g., system 100) such as by an IMD (e.g., IMD 102 of FIG. 1). Steps of method 600 may be stored as instructions at a memory of the IMD (e.g., the memory 114 of FIG. 1). The instructions may be executable by one or more processors (e.g., processors of the IMD 102, the controller 114 of FIG. 1, or other logic) to perform closed loop stimulation in connection with managing a patient's pain in accordance with the concepts disclosed herein.

Method 600 includes, at block 602, delivering, via a second plurality of electrodes (e.g., second plurality of electrodes 214, 304 of FIGS. 2 and 3), first one or more stimulation pulses to the DRG of the patient. At block 604, method 600 includes sensing, via a first plurality of electrodes (e.g., first plurality of electrodes 212, 302 of FIGS. 2 and 3), first ENG data corresponding to first neuronal activity of first neural tissue disposed between the DRG and the spinal cord of the patient and adjacent to the DRG of the patient (e.g., corresponding to tissue of central process 206 of FIG. 2). Moreover, at block 606, method 600 includes sensing, via a third plurality of electrodes (e.g., third plurality of electrodes 216, 306 of FIGS. 2 and 3), second ENG data corresponding to second neuronal activity of second neural tissue disposed away from the DRG and adjacent to the spinal nerve from which the DRG emerges (e.g., corresponding to tissue of peripheral process 204 of FIG. 2). At block 608, a blocking effect of the first one or more stimulation pulses delivered to the DRG of the patient based on the first ENG data and the second ENG data is estimated (e.g., by controller 110 of FIG. 1). At block 610, method 600 includes generating, by an IPG (e.g., IPG 104 of FIG. 1), second one or more stimulation pulses based on the estimated blocking effect and at block 612, applying the second one or more stimulation pulses to the DRG using one or more electrodes of the second plurality of electrodes (e.g., second plurality of electrodes 214, 304 of FIGS. 2 and 3).

In an example of method 600 and referring to FIGS. 1-3, one or more electrodes of first plurality of electrodes 212, 302 of FIGS. 2 and 3 may be configured to sense the first ENG data while one or more electrodes of third plurality of electrodes 216, 306 of FIGS. 2 and 3 may be configured to sense second ENG data. Thus, the first ENG data may correspond to pain signals detected at central process 206, and the second ENG data may contain pain signals detected at peripheral process 204. Controller 110 of FIG. 1 may be configured to compare first ENG data to second ENG data to determine an effectiveness of the first one or more stimulation pulses in blocking propagation of pain signals (e.g., propagation of pain signals from the peripheral process to the central process of the nociceptive neurons) of the patient. The difference between the first and second ENG data may indicate a reduction of pain signals and provide a mechanism for characterizing the efficacy of the stimulation pulses. Controller 110 may be configured to estimate, based on comparison of the first ENG data to the second ENG data, a blocking effect of the stimulation pulses delivered to DRG 202 in the epineural tissue of the patient via first plurality of electrodes 212, 302. In particular, an estimate of the blocking effect may be based on a difference between the first ENG data and the second ENG data.

It is noted that the above-described ENG data may contain both pain signals and somatosensory signals. The somatosensory signals are considered to have fast temporal variation while the nociceptive pain signals are more consistent. For example, when an object touches skin, there is increased ENG signal during the touch, but the ENG signal disappears with the removal of the object (e.g., due somatosensory signals). In contrast, chronic neuropathic pain signals are more consistent with persistent increased neuronal activity. Utilizing these temporal features or characteristics, the pain related information can be extracted from the ENG data. Different analytical methods (e.g., independent component analysis, wavelet transform, and multitaper methods) may be used to extract features (e.g., pain signals) from the ENG data and perform analysis. Machine learning algorithms may also be incorporated to increase the accuracy of feature extraction, analysis, or both.

Additionally, at block 608 of method 600, estimating a blocking effect of the first one or more stimulation pulses delivered to the DRG of the patient based on the first ENG data and the second ENG data may provide an indication of how effective the stimulation pulses are at mitigating pain of the patient. For example and as explained above, a difference between the first ENG data to the second ENG data may be determined (e.g., by controller 110 of FIG. 1). The parameters used to generate subsequent stimulation pulses delivered to the DRG may be modified to enhance the blocking effect caused by stimulation of the DRG. As explained above, the blocking effect arises when the DRG is stimulated and prevents or reduces propagation of the pain signals received at the peripheral process to the central process and therefore, the patient's brain, which reduces the patient's perceived pain.

In the context of FIGS. 1-3, controller 110 may be configured to modify one or more of the stimulation parameters. For instance, controller 110 may be configured to modify the one or more stimulation parameters based on the estimated blocking effect to generate the modified set of stimulation parameters. Additionally, controller 110 may be configured to cause IPG 104 to generate second one or more stimulation pulses in accordance with the one or more modified stimulation parameters. The modified set of stimulation parameters may be configured to increase the blocking effect achieved by additional stimulation pulses delivered to DRG 202 via second plurality of electrodes 214, 304 of FIGS. 2 and 3. An increase in the blocking effect may correlate with an increased mitigation of the pain of the patient. To illustrate, by further attenuating (e.g., blocking) propagation of pain signals from the peripheral process to the central process and then to the patient's spine and brain via stimulation of DRG 202, an extent or a degree pain experienced by the patient is diminished.

As described above with reference to method 400 of FIG. 4 and in an example, method 600 may further include determining, by the controller (e.g., controller 110), one or more metrics based on feedback data that includes the first ENG data, the second ENG data, or both and received at the controller. The one or more metrics may include a noise metric indicative of distortions in the first ENG data, the second ENG data, or both. The distortions may be attributable to interference induced in one or more electrodes of the first plurality of electrodes (e.g., first plurality of electrodes 212, 302 of FIGS. 2 and 3), of the third plurality of electrodes (e.g., third plurality of electrodes 216, 306), or both and caused by delivering stimulation pulses to the DRG (or from other sources). The first operating mode includes sensing ENG data by one or more electrodes of the first plurality of electrodes (e.g., first plurality of electrodes 212, 302), one or more electrodes of the third plurality of electrodes (e.g., third plurality of electrodes 216, 306), or both at least partially simultaneously with applying stimulation pulses to the DRG. The second operating mode may include sensing the ENG data in between applying the stimulation pulses to the DRG. Further, controller 110 may be configured to switch from the first operating mode to the second operating mode based on the noise metric satisfying a threshold value (e.g., stored in memory 114 of FIG. 1). For example, in response to the noise metric exceeding the threshold value (indicating excessive interference with sensing operations), controller 110 may be configured to switch from the first operating mode to the second operating mode (or maintain operation in the IMD 102 in the second operating mode if currently operating in the second operating mode). Additionally, controller 110 may switch from the second operating mode to the first operating mode (or maintain operation in the IMD 102 in the first operating mode if currently operating in the first operating mode) in response to the noise metric not exceeding the threshold value.

As another example, method 600 may include iteratively sensing ENG data, estimating blocking effects based on the ENG data, generating one or more stimulation pulses based on the blocking effects, and applying one or more stimulation pulses until an estimated pain level value of the patient satisfies a threshold value (e.g., a threshold value stored in memory 114 of FIG. 1). Additionally, method 600 may include determining, by a controller (e.g., controller 110 of FIG. 1), that the estimated pain level value of the patient satisfies the threshold value. The estimated pain level value of the patient may be determined based on the ENG data. For example, by iteratively comparing first ENG data and second ENG data after iteratively delivering one or more stimulation pulses, controller 110 may be configured to determine that the pain level of the patient satisfies the threshold value, denoting an acceptable level (e.g., a low level) of pain, or continue to adjust the stimulation parameters until the acceptable level of pain is achieved.

Referring to FIG. 7, a flowchart depicting another exemplary method for managing patient pain via closed loop stimulation according to embodiments of the present disclosure is shown as method 700. In an aspect, method 700 may be performed by one or more components of a neurostimulation system (e.g., system 100) such as by an IMD (e.g., IMD 102 of FIG. 1). Steps of method 700 may be stored as instructions at a memory of the IMD (e.g., the memory 114 of FIG. 1). The instructions may be executable by one or more processors (e.g., processors of the IMD 102, the controller 114 of FIG. 1, or other logic) to perform closed loop stimulation in connection with managing a patient's pain in accordance with the concepts disclosed herein.

At block 702, method 700 includes sensing, by a first plurality of electrodes (e.g., first plurality of electrodes 212, 302 of FIGS. 2 and 3), evoked compound action potential (ECAP) signals corresponding to neural tissue disposed between the DRG and a spinal cord of the patient (e.g., at central process of pseudo-unipolar neurons 206 of FIG. 2). In an aspect, the ECAP signals may be induced by one or more stimulation pulses delivered by the third plurality of electrodes. The ECAP data may be indicative of an effectiveness of the stimulation pulses in blocking action potential (AP) propagation of pain signals at the DRG. At block 704, a blocking effect of the stimulation pulses delivered to the DRG by a second plurality of electrodes (e.g., second plurality of electrodes 214, 304 of FIGS. 2 and 3) is estimated based on the ECAP data corresponding to the ECAP signals recorded by a first plurality of electrodes (e.g., first plurality of electrodes 212, 302 of FIGS. 2 and 3). Further, method 700 includes, at block 706, generating third one or more stimulation pulses using the IPG (e.g., IPG 104) based on sensing the ECAP signals and, at block 708, applying the third one or more stimulation pulses to the DRG via one or more of the second plurality of electrodes.

In an aspect, the ECAP data may be recorded or captured simultaneously with delivery of stimulation pulses or stimulation pulse trains to the DRG. In an additional or alternative aspect, the ECAP data may be captured at the central process of the pseudo-unipolar neurons in between delivery of stimulation pulses or stimulation pulse trains to the DRG. Capturing the ECAP data in between delivery of stimulation pulses or pulse trains may minimize the impact of stimulation artifacts. In yet another additional or alternative aspect, a determination may be made regarding a signal quality associated with the ECAP data, such as to determine whether the ECAP data is being degraded by stimulation artifacts. When stimulation artifacts are degrading the ECAP data, recording of the ECAP data may be switched from being captured simultaneously with delivery of the stimulation pulses or pulse trains to being recorded in between stimulation pulses or pulse trains. Moreover, in other aspects, ECAP data may also be recorded at the peripheral process in response to delivery of stimulation pulses to the peripheral process in addition to the ECAP signal recording at the central process.

It is noted that a magnitude or amplitude of an ECAP waveform corresponding to the ECAP signals may be greater than a magnitude or amplitude of an ENG waveform. Accordingly, a first blocking effect estimate determined (e.g., at controller 110 of FIG. 1) based on ECAP data, is expected to have higher SNR than a second blocking effect estimate determined based on ENG data. Further, since ECAP data generally has a higher amplitude than ENG data, the ECAP data may be less subject to degradation from stimulation artifacts or other noise as compared to the ENG data and hence be detected more easily.

Further, in aspects, method 700 may include, prior to sensing ECAP signals, applying, by second one or more electrodes of the third plurality of electrodes (e.g., third plurality of electrodes 216, 306 of FIGS. 2 and 3), one or more stimulation pulses to the neural tissue disposed away from the DRG and adjacent to the spinal nerve distal to the DRG (e.g., peripheral process 204). Applying the one or more stimulation pulses in this manner may excite sensory axons, thereby enabling determination (e.g., at controller 110 of FIG. 1) of one or more characteristics associated with neural activity based on the ECAP data. In an aspect, the one or more characteristics may include information associated with one or more fiber types. Activation of the C-fibers or Aδ fibers requires high intensity stimulation. A profile of the action potential propagation speed may be used to determine the fiber types from the ECAP data. Moreover, method 700 may include estimating an efficacy of the stimulation pulses based on the one or more characteristics. The efficacy of the stimulation pulses may indicate how effective the stimulation pulses are at mitigating patient pain and may be determined by quantifying a blocking effect achieved by the stimulation. In an aspect, the blocking effect may be estimated based on an Aβ fiber blocking percentage using the ECAP data, which may be derived from the fiber types identified or determined based on the ECAP data. To illustrate, an Aβ fiber blocking percentage may be included in or determined based on the one or more characteristics and the efficacy may be estimated based on the Aβ fiber blocking percentage.

In another aspect and in accordance with method 700, generating stimulation pulses (e.g., at block 706) may include modifying, by a controller (e.g., controller 110 of FIG. 1), one or more stimulation parameters (e.g., parameters 116). For example, controller 110 may be configured to modify the one or more stimulation parameters based, at least in part, on the ECAP data or information derived from the ECAP data (e.g., an estimated blocking effect or blocking percentage of Aβ fibers caused by the stimulation pulses). Modifying the one or more stimulation parameters may be configured to enhance a blocking effect and attenuate propagation of pain signals emanating from the DRG. For example, as the parameters of the one or more stimulation pulses are modified (e.g., by controller 110 of FIG. 1), such as by increasing an amplitude of the one or more stimulation pulses, a blocking percentage of Aβ fibers may increase. The parameters of the one or more stimulation pulses (e.g., stimulation amplitude) may be adjusted to correspond to a certain blocking percentage of Aβ fibers as determined by a change in an amplitude of ECAP data recorded from the dorsal root or rootlets. The modified set of stimulation parameters may be configured to increase the blocking effect of the additional stimulation pulses delivered to the DRG (e.g., DRG 202 of FIG. 2), which, likewise, translates into an increased mitigation of pain of the patient. It is to be understood that the ECAP data may be indicative of an efficacy (e.g., an effectiveness of blocking pain signals of the patient) of stimulation pulses applied to DRG.

Referring to FIG. 8, a flowchart depicting another exemplary method for managing patient pain via closed loop stimulation according to embodiments of the present disclosure is shown as method 800. In an aspect, method 800 may be performed by one or more components of a neurostimulation system (e.g., system 100) such as by an IMD (e.g., IMD 102 of FIG. 1). Steps of method 800 may be stored as instructions at a memory of the IMD (e.g., the memory 114 of FIG. 1). The instructions may be executable by one or more processors (e.g., processors of the IMD 102, the controller 114 of FIG. 1, or other logic) to perform closed loop stimulation in connection with managing a patient's pain in accordance with the concepts disclosed herein.

Method 800 includes selecting, at block 802, by a controller (e.g., controller 110 of FIG. 1) of an IMD (e.g., IMD 102 of FIG. 1), a therapy modality for conducting a closed-loop neurostimulation therapy. The IMD is programmed to perform a plurality of closed-loop neurostimulation therapies, such as those described above, and the therapy modality may be selected from among the plurality of closed-loop neurostimulation therapies programmed for the IMD. Additionally, method 800 includes, at block 804, generating one or more stimulation pulses using an IPG (e.g., IPG 104 of FIG. 1) and based on the selected therapy modality. Moreover, method 800 includes, at block 806, applying, via one or more electrodes (e.g., first plurality of electrodes 212, 302, second plurality of electrodes 214, 304, and/or third plurality of electrodes 216, 306), the one or more stimulation pulses to neural tissue of the patient.

In aspects, the plurality of closed-loop neurostimulation therapies include at least a first therapy modality corresponding to method 500 of FIG. 5 and a second therapy modality corresponding to method 600 of FIG. 6. In additional or alternative aspects, the plurality of closed-loop neurostimulation therapies include at least a first therapy modality corresponding to method 600 of FIG. 6 and a second therapy modality corresponding to method 700 of FIG. 7. In additional or alternative aspects, the plurality of closed-loop neurostimulation therapies include at least a first therapy modality corresponding to method 500 of FIG. 5 and a second therapy modality corresponding to method 700 of FIG. 7. In additional or alternative aspects, the plurality of closed-loop neurostimulation therapies include at least a first therapy modality corresponding to method 500 of FIG. 5, a second therapy modality corresponding to method 600 of FIG. 6. In additional or alternative aspects, and a third therapy modality corresponding to method 700 of FIG. 7. Furthermore, it is noted that an IMD operating in accordance with method 800 may be configured to provide more than three therapy modalities and any of the configured therapy modalities may be selected for use in treating patient pain according to the configuration of the IMD and the available therapy modalities.

As can be appreciated, any combination and temporal sequencing of the therapy modalities can be selected to enhance pain mitigation. For instance, the controller (e.g., controller 110) may be configured to select the first therapy modality and the third therapy modality, applying each in tandem or sequentially to reduce pain of the patient. Alternatively or additionally, the controller may be configured to select the first therapy modality, the second therapy modality, and the third therapy modality, applying each in tandem or in sequence (e.g., applying the second therapy modality, then the first therapy modality, and then the third therapy modality). In aspects, method 800 may include selecting (e.g., by a controller such as controller 110 of FIG. 1) the first therapy modality. Method 800 may further include estimating, by the controller, a blocking effectiveness of the first therapy modality. The blocking effectiveness may correspond to an effectiveness of blocking AP propagation of pain signals to the spinal cord of the patient. In response to determining that the blocking effectiveness of the first therapy modality fails to satisfy a threshold value, method 800 may include selecting, by the controller, the second therapy modality. In response to determining, by the controller, that the blocking effectiveness of the second therapy modality fails to satisfy the threshold value, method 800 may additionally include selecting, by the controller, the third therapy modality. Additionally, input from other sensors, such as accelerometers, gyroscopes, etc., as mentioned with reference to FIGS. 1 and 5 can be incorporated into any of the aforementioned therapy modalities to enhance an effectiveness of pain management.

FIGS. 9A and 9B illustrate a process for manufacturing a lead having high density electrodes for sensing and/or stimulation according to embodiments of the present disclosure. Steps of the foregoing process are described in FIG. 9A, while FIG. 9B illustrates the corresponding steps identified in FIG. 9A. At block 902A of FIG. 9A, openings 902B may be created in a multi-lumen tubing 904B. The multi-lumen tubing 904B may be formed from a biocompatible electrically insulating material 906B, such as biocompatible polymer, as illustrated in FIG. 9B. At 904A of FIG. 9A, conductive material 908B of FIG. 9B, may be threaded through multi-lumen tubing 904B. Conductive material 908B may be conductive cabling or wires. At 906A, electrode material 910B may be deposited onto conductive material 908B as depicted in FIG. 9B. The electrode material 910B may be deposited through sputter deposition, chemical vapor deposition (CVD), three dimensional (3D) printing, and/or other methods. In embodiments, electrode material 910B may be the same material as conductive material 908B; however, in other embodiments, electrode material 910B may be a different material from conductive material 908B. As illustrated at block 908A, in some aspects the electrode may be formed by applying electrode material 910B and connecting electrode material 910B to conductors 908B using one or more conductive vias 922, as more clearly shown in insert 920. In some aspects, a biocompatible insulator may be deposited on electrode material 910B to tailor a geometry of resulting electrode 914B. For example, by precisely controlling deposition of biocompatible insulator 906B onto electrode material 910B, it is possible to generate different types of electrode configurations and geometries.

Referring to FIGS. 10A and 10B, an alternative process for manufacturing a lead having high density electrodes for sensing and/or stimulation according to embodiments of the present disclosure is shown. Steps of the foregoing process are described in FIG. 10A, while FIG. 10B illustrates the state of the lead for the corresponding steps identified in FIG. 10A. At block 1002A, openings 1002B may be created in a multi-lumen tubing 1004B, the multi-lumen tubing 1004B being fashioned from biocompatible electrically insulating material 1006B, such as biocompatible polymer, as illustrated in FIG. 10B. At block 1004A, conductive material 1008B of FIG. 10B, may be threaded through multi-lumen tubing 1004B. Conductive material 1008B may be conductive cabling or wires, as described above. When threaded through multi-lumen tubing 1004B, conductive material may be threaded or routed out of openings 1002B and back into an interior portion of multi-lumen tubing 1004B via other ones of openings 1002B, as shown in 10B (and corresponding block 1004A). At block 1006A, multi-lumen tubing 1004B and the conductive cabling formed from conductive material 1008B are compressed to form a lead body of a lead having a plurality of conductive openings as depicted in FIG. 10B. In some aspects, filler (e.g., insulative filler, conductive filler, or both) may be deposited in areas where conductive material 1008B is exposed in order to smoothen the surface of the lead body, configure the geometry of the electrodes, or other purposes.

The manufacturing processes of FIGS. 9A-10B may be utilized to create leads having high electrode densities suitable for use in performing closed loop stimulation to treat and manage patient pain in accordance with aspects of the present disclosure. However, it is to be understood that the manufacturing processes of FIGS. 9A-10B have been shown for purposes of illustration, rather than by way of limitation, and the closed loop stimulation systems for managing patient pain in accordance with the concepts disclosed herein may readily utilize leads manufactured using other techniques or combinations of the above-described techniques and other techniques. As shown above, the closed loop systems and techniques described herein enable recording of feedback data that may be reliably used to detect biomarkers indicative of patient pain. The ability to reliably detect such biomarkers enables closed loop stimulation systems to be applied to treat patient pain in more robust manner than prior systems, which were limited to one or more static stimulation configurations and could result in patient discomfort due to overstimulation or understimulation. For example, the disclosed biomarker detection and analysis techniques may be applied to closed loop stimulation systems to dynamically adjust stimulation therapy based on observed patient pain levels, resulting in effective mitigation of patient pain without overstimulation or understimulation and the discomfort associated therewith. It is noted that in some aspects, the above-described techniques of FIGS. 4-8 may be utilized with additional types of feedback data, such as patient mobility data (e.g., sensor data from an accelerometer, gyroscope, etc.) or other types of sensor data (e.g., heart rate, blood pressure, etc.), and the management of patient pain may be based on the above-described techniques and the other types of feedback data. Taking the other types of feedback data into account may be beneficial as it allows additional types of information to be taken into account when managing the patient's perceived pain, such as whether the patient is running, walking, etc.

Additionally, it is noted that the exemplary closed-loop techniques of FIGS. 4-8 may be used to manage patient pain individually, or in combination. For example, the feedback-based technique of FIG. 7, which utilizes ECAP data, may be performed as a primary pain indication, but the techniques of FIGS. 5 and/or 6 may be utilized to periodically validate the effectiveness of the ECAP-based technique, such as to measure the blocking effect using method 600 of FIG. 6, or evaluating whether hyperactivity is present, as in method 500 of FIG. 5. In some aspects, the different techniques of FIGS. 4-8 may be associated with different power consumption levels and an IMD (e.g., the IMD 102 of FIG. 1) may be configured to select a particular mode of operation in accordance with one of techniques of FIGS. 4-8 based on a power level of the IMD's battery. This may improve the battery life of the IMD, allowing the patient's pain to be effectively managed for a prolonged period of time before battery charging (or replacement) is required.

Furthermore, it is noted that when adjusting the stimulation parameters based on the feedback data, IMDs operating in accordance with the concepts disclosed herein may be configured to make multiple adjustments to the stimulation parameters and monitor feedback data to evaluate how effective each adjustment is at mitigating patient pain. For example, stimulation pulses having different amplitudes may be delivered sequentially upon making a determination to modify the stimulation parameters and feedback data may be obtained for each different amplitude. The amplitude associated with feedback data indicating a higher effectiveness at blocking patient pain perception may be selected for use in treating the patient's pain. Subsequently, situations may arise where the parameter(s) selected as providing the best improvement to the patient's pain may no longer be effective (e.g., based on analysis of the feedback data using the techniques described above), such as due to a different state of the patient (e.g., running, walking, standing, laying down, etc., or other reasons), and a different set of adjustments may be made to the stimulation parameters. Furthermore, it is noted that while the non-limiting example described immediately above involves titrating different amplitude levels and then selecting one for use in treating the patient's pain, any stimulation parameters may be similarly adjusted and evaluated, individually or in combination with other parameters, to identify a set of stimulation parameters providing improved mitigation of patient pain in accordance with the concepts disclosed herein.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Components, the functional blocks, and the modules described herein with respect to FIGS. 1-10B) include processors, electronics devices, hardware devices, electronics components, logical circuits, memories, software codes, firmware codes, among other examples, or any combination thereof. In addition, features discussed herein may be implemented via specialized processor circuitry, via executable instructions, or combinations thereof.

Those of skill would further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various aspects of the present disclosure may be combined or performed in ways other than those illustrated and described herein.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules, and circuits described in connection with the aspects disclosed herein may be implemented or performed with a general purpose single-or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or any conventional processor, controller, microcontroller, or state machine. In some implementations, a processor may also be implemented as a combination of computing devices, such as a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or any combination thereof. Implementations of the subject matter described in this specification also may be implemented as one or more computer programs, that is one or more modules of computer program instructions, encoded on a computer storage media for execution by, or to control the operation of, data processing apparatus.

If implemented in software, the functions may be stored on or transmitted over as one or more instructions or code on a computer-readable medium. The processes of a method or algorithm disclosed herein may be implemented in a processor-executable software module which may reside on a computer-readable medium. Computer-readable media includes both computer storage media and communication media including any medium that may be enabled to transfer a computer program from one place to another. A storage media may be any available media that may be accessed by a computer. By way of example, and not limitation, such computer-readable media can include random-access memory (RAM), read-only memory (ROM), electrically erasable programmable read-only memory (EEPROM), CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, hard disk, solid state disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to some other implementations without departing from the spirit or scope of this disclosure. Thus, the claims are not intended to be limited to the implementations shown herein, but are to be accorded the widest scope consistent with this disclosure, the principles and the novel features disclosed herein.

Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of any device as implemented.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, some other implementations are within the scope of the following claims. In some cases, the actions recited in the claims may be performed in a different order and still achieve desirable results.

As used herein, including in the claims, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. the term “or,” when used in a list of two or more items, means that any one of the listed items may be employed by itself, or any combination of two or more of the listed items may be employed. For example, if a composition is described as containing components A, B, or C, the composition may contain A alone; B alone; C alone; A and B in combination; A and C in combination; B and C in combination; or A, B, and C in combination. Also, as used herein, including in the claims, “or” as used in a list of items prefaced by “at least one of” indicates a disjunctive list such that, for example, a list of “at least one of A, B, or C” means A or B or C or AB or AC or BC or ABC (that is A and B and C) or any of these in any combination thereof. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed aspect, the term “substantially” may be substituted with “within [a percentage] of” what is specified, where the percentage includes 0.1, 1, 5, and 10 percent; and the term “approximately” may be substituted with “within 10 percent of” what is specified. The phrase “and/or” means and or.

Although the aspects of the present disclosure and their advantages have been described in detail, it should be understood that various changes, substitutions and alterations can be made herein without departing from the spirit of the disclosure as defined by the appended claims. Moreover, the scope of the present application is not intended to be limited to the particular implementations of the process, machine, manufacture, composition of matter, means, methods and processes described in the specification. As one of ordinary skill in the art will readily appreciate from the present disclosure, processes, machines, manufacture, compositions of matter, means, methods, or operations, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or operations.

What is claimed is:

1. A method of providing a neurostimulation therapy to a patient using a neurostimulation system, wherein the neurostimulation system is implanted in the patient and comprises: (1) a stimulation lead with a first plurality of electrodes, a second plurality of electrodes, and a third plurality of electrodes, wherein the second plurality of electrodes is disposed adjacent to a dorsal root ganglion (DRG) of the patient, the first plurality of electrodes is disposed between the DRG and a spinal cord of the patient and adjacent to dorsal root and rootlets of the patient, and the third plurality of electrodes is disposed away from the DRG and adjacent to a spinal nerve distal to the DRG; and (2) an implantable pulse generator (IPG), wherein the method comprises:
sensing, via the second plurality of electrodes, activity of nociceptive neurons of the DRG, the activity of the nociceptive neurons indicative of neuropathic pain of the patient;
switching, by a controller, an operating mode of the neurostimulation system between a first operating mode and a second operating mode based on the sensing, the first operating mode comprising sensing the activity of the nociceptive neurons at least partially simultaneously with delivery of one or more stimulation pulses to the patient and the second operating mode comprising performing the sensing in between delivery of the one or more stimulation pulses to the patient;
generating, by the IPG, the one or more stimulation pulses; and
applying the one or more stimulation pulses in accordance with the first operating mode or the second operating mode.

2. The method of claim 1, wherein:
applying the one or more stimulation pulses comprises:
in the first operating mode, applying the one or more stimulation pulses via first one or more electrodes of the second plurality of electrodes, and wherein the sensing is performed at least partially simultaneously with the applying of the one or more stimulation pulses using second one or more electrodes of the second plurality of electrodes, and
in the second operating mode, applying the one or more stimulation pulses via the first one or more electrodes of the second plurality of electrodes, wherein the sensing is performed using the second one or more electrodes of the second plurality of electrodes subsequent to the applying.

3. The method of claim 1, further comprising:
receiving, by the controller, feedback data comprising neuronal data corresponding to the activity of the nociceptive neurons; and
determining, by the controller:
one or more metrics based on the feedback data, wherein the one or more metrics comprise a neuronal activity level metric and a noise metric,
whether to modify one or more stimulation parameters corresponding to a therapy regimen based on whether the neuronal activity level metric satisfies a first threshold level, and
whether to switch the operating mode between the first operating mode and the second operating mode based on whether the noise metric satisfies a second threshold level.

4. The method of claim 3, further comprising modifying, by the controller, at least one stimulation parameter of the one or more stimulation parameters in response to determining that the neuronal activity level metric satisfies the first threshold level, wherein the one or more stimulation pulses are generated according to at least one modified stimulation parameter.

5. The method of claim 3, further comprising switching, by the controller, from the first operating mode to the second operating or from the second operating mode to the first operating mode in response to determining that the noise metric satisfies the second threshold level.

6. The method of claim 1, wherein:
a first threshold distance separates the first plurality of electrodes from the second plurality of electrodes,
a second threshold distance separates the third plurality of electrodes from the second plurality of electrodes, and
the first threshold distance and the second threshold distance are configured to reduce noise sensed at first one or more electrodes of the first plurality of electrodes, the second plurality of electrodes, or the third plurality of electrodes during sensing by some electrodes when other electrodes deliver the one or more stimulation pulses to neural tissue of the patient.

7. The method of claim 1, further comprising:
sensing, via one or more sensors disposed along the stimulation lead, in the neurostimulation system, or in both, a state of the patient; and
modifying, by the controller, one or more stimulation parameters based on feedback data comprising state data obtained based on sensing the state of the patient, wherein the one or more stimulation pulses are generated based on one or more modified stimulation parameters.

8. A method of providing a neurostimulation therapy to a patient using a neurostimulation system, wherein the neurostimulation system is implanted in the patient and comprises: (1) a stimulation lead with a first plurality of electrodes, a second plurality of electrodes, and a third plurality of electrodes, wherein the second plurality of electrodes is disposed adjacent to a dorsal root ganglion (DRG) of the patient, the first plurality of electrodes is disposed between the DRG and a spinal cord of the patient and adjacent dorsal root and rootlets of the patient, and the third plurality of electrodes is disposed away from the DRG and adjacent to a spinal nerve distal to the DRG; and (2) an implantable pulse generator (IPG), wherein the method comprises:
delivering, via the second plurality of electrodes, first one or more stimulation pulses to the DRG of the patient, wherein the first one or more stimulation pulses are generated by the IPG based on stimulation parameters configured to mitigate pain of the patient;
sensing, via the first plurality of electrodes, first electroneurogram data corresponding to first neuronal activity of first neural tissue disposed between the DRG and the spinal cord of the patient;
sensing, via the third plurality of electrodes, second electroneurogram data corresponding to second neuronal activity of second neural tissue disposed away from the DRG and adjacent to the spinal nerve;
estimating, by a controller, a pain blocking effect of the first one or more stimulation pulses delivered to the DRG of the patient based on the first electroneurogram data and the second electroneurogram data;
generating second one or more stimulation pulses using the IPG and based on the pain blocking effect; and
applying, via the second plurality of electrodes, the second one or more stimulation pulses to the DRG.

9. The method of claim 8, wherein sensing the first electroneurogram data occurs while sensing the second electroneurogram data, and wherein estimating the blocking effect of the first one or more stimulation pulses comprises comparing, by the controller, the first electroneurogram data and the second electroneurogram data to determine an effectiveness of the first one or more stimulation pulses in blocking action potential (AP) propagation of pain signals from the DRG to spinal nerve tissue of the patient.

10. The method of claim 8, wherein generating the second one or more stimulation pulses comprises:
modifying, by the controller, one or more stimulation parameters; and
generating the second one or more stimulation pulses in accordance with the one or more modified stimulation parameters.

11. The method of claim 10, further comprising:
iteratively performing the sensing, the estimating, the modifying, and the generating and applying of stimulation pulses until an estimated pain level of the patient satisfies a threshold pain level.

12. The method of claim 8, further comprising determining, by the controller, one or more metrics based on the first electroneurogram data, the second electroneurogram data, or both, wherein the one or more metrics comprise a noise metric indicative of distortions in the first electroneurogram data, the second electroneurogram data, or both, the distortions attributable to interference impacting the first plurality of electrodes, the third plurality of electrodes, or both and caused by delivery of the first one or more stimulation pulses to the DRG.

13. The method of claim 12, further comprising switching, by the controller, an operating mode of the neurostimulation system between a first operating mode and a second operating mode based on the noise metric, wherein:
the first operating mode comprises sensing electroneurogram data via the first plurality of electrodes and the third plurality of electrodes at least partially simultaneously with applying stimulation pulses to the DRG via the second plurality of electrodes, and
the second operating mode comprises sensing the electroneurogram data via the first plurality of electrodes and the third plurality of electrodes in between applying the stimulation pulses to the DRG via the second plurality of electrodes.

14. The method of claim 13, further comprising determining, by the controller, to switch between the first operating mode to the second operating mode based on the noise metric satisfying a threshold value.

15. A method of providing a neurostimulation therapy to a patient using a neurostimulation system, wherein the neurostimulation system is implanted in the patient and comprises: (1) a stimulation lead with a first plurality of electrodes, a second plurality of electrodes, and a third plurality of electrodes, wherein the second plurality of electrodes is disposed adjacent to a dorsal root ganglion (DRG) of the patient, the first plurality of electrodes is disposed between the DRG and a spinal cord of the patient and adjacent to dorsal root and rootlets of the patient, and the third plurality of electrodes is disposed away from the DRG and adjacent to a spinal nerve distal the DRG; and (2) an implantable pulse generator (IPG), wherein the method comprises:
sensing, by the third plurality of electrodes, evoked compound action potential (ECAP) signals corresponding to neural tissue disposed between the DRG and the spinal cord of the patient;
estimating, based on ECAP data corresponding to the ECAP signals, a blocking effect of stimulation pulses delivered to the DRG by the second plurality of electrodes;
generating additional stimulation pulses based on the ECAP data; and
applying the additional stimulation pulses to the DRG via one or more of the second plurality of electrodes.

16. The method of claim 15, wherein the ECAP data is indicative of an effectiveness of the stimulation pulses in blocking action potential (AP) propagation of pain signals originating at the DRG.

17. The method of claim 15, wherein a signal to noise ratio (SNR) of an ECAP signal is greater than a SNR of an electroneurogram data, and wherein a first blocking effect estimate, generated based on ECAP data, is more accurate than a second blocking effect estimate, generated based on electroneurogram data.

18. The method of claim 15, further comprising, when sensing the ECAP signals, applying, by the third plurality of electrodes, one or more stimulation pulses to the neural tissue disposed away from the DRG and adjacent to the spinal nerve, wherein applying the one or more stimulation pulses is configured to excite sensory axons.

19. The method of claim 15, wherein generating the additional stimulation pulses comprises modifying, by a controller of the neurostimulation system, one or more stimulation parameters associated with the additional stimulation pulses, wherein modifying the one or more stimulation parameters is configured to enhance blocking a propagation of pain signals.

20. A method of providing a neurostimulation therapy to a patient using a neurostimulation system, wherein the neurostimulation system is implanted in the patient and comprises: (1) a stimulation lead with a first plurality of electrodes, a second plurality of electrodes, and a third plurality of electrodes, wherein the second plurality of electrodes is disposed adjacent to a dorsal root ganglion (DRG) of the patient, the first plurality of electrodes is disposed between the DRG and a spinal cord of the patient and adjacent to dorsal root and rootlets of the patient, and the third plurality of electrodes is disposed away from the DRG and adjacent to a spinal nerve distal to the DRG; and (2) an implantable pulse generator (IPG), wherein the method comprises:
selecting, by a controller of an implanted medical device (IMD), a therapy modality for conducting a closed-loop neurostimulation therapy, wherein the IMD is programmed to perform a plurality of closed-loop neurostimulation therapies and the therapy modality is selected from among the plurality of closed-loop neurostimulation therapies programmed for the IMD, wherein the plurality of closed-loop neurostimulation therapies include at least:
a first therapy modality in which one or more stimulation pulses are delivered to the DRG via the second plurality of electrodes based on sensed activity of nociceptive neurons;
a second therapy modality in which the one or more stimulation pulses are delivered to the DRG, via the second plurality of electrodes based on first electroneurogram data associated with first neural tissue disposed between the DRG and the spinal cord of the patient and adjacent to the dorsal root and rootlets of the patient and second electroneurogram data associated with second neural tissue disposed away from the DRG and adjacent to the spinal nerve, and
a third therapy modality in which the stimulation pulses are delivered to the DRG via the second plurality of electrodes based on evoked compound action potential (ECAP) data corresponding to ECAP signals; and
generating the one or more stimulation pulses using the IPG and based on the selected therapy modality; and
applying, via one or more electrodes, the one or more stimulation pulses to neural tissue of the patient.
